SEQUENCE LISTING (22 sequences)

SEQ ID NO: 1 - DNA, 681 bp, Artificial
  synthetic
atgggcgtgatcaagcccgacatgaagatcaagctgcggatggagggcgccgtgaacggccacaaattcgtgatcgagggcgacgggaaaggcaagccctttgagggtaagcagactatggacctgaccgtgatcgagggcgcccccctgcccttcgcttatgacattctcaccaccgtgttcgactacggtaaccgtgtcttcgccaagtaccccaaggacatccctgactacttcaagcagaccttccccgagggctactcgtgggagcgaagcatgacatacgaggaccagggaatctgtatcgctacaaacgacatcaccatgatgaagggtgtggacgactgcttcgtgtacaaaatccgcttcgacggggtcaacttccctgctaatggcccggtgatgcagcgcaagaccctaaagtgggagcccagtaccgagaagatgtacgtgcgggacggcgtactgaagggcgatgttaatatggcactgctcttggagggaggcggccactaccgctgcgacttcaagaccacctacaaagccaagaaggtggtgcagcttcccgactaccacttcgtggaccaccgcatcgagatcgtgagccacgacaaggactacaacaaagtcaagctgtacgagcacgccgaagcccacagcggactaccccgccaggccggc

SEQ ID NO: 2 - protein, 227 aa, Artificial
  synthetic
MGVIKPDMKIKLRMEGAVNGHKFVIEGDGKGKPFEGKQTMDLTVIEGAPLPFAYDILTTVFDYGNRVFAKYPKDIPDYFKQTFPEGYSWERSMTYEDQGICIATNDITMMKGVDDCFVYKIRFDGVNFPANGPVMQRKTLKWEPSTEKMYVRDGVLKGDVNMALLLEGGGHYRCDFKTTYKAKKVVQLPDYHFVDHRIEIVSHDKDYNKVKLYEHAEAHSGLPRQAG

SEQ ID NO: 3 - DNA, 726 bp, Artificial
  synthetic
tcgacccctaaggaggccaccatgagcgtgatcaagcccgacatgaagatcaagctgcgcatggagggcgccgtgaacggccacaagttcgtgatcgagggcgacggcaagggcaagcccttcgagggcaagcagaccatggacctgaccgtgatcgagggcgcccccctgcccttcgcctacgacatcctgaccaccgtgttcgactacggcaaccgcgtgttcgccaagtaccccaaggacatccccgactacttcaagcagaccttccccgagggctacagctgggagcgcagcatgacctacgaggaccagggcatctgcatcgccaccaacgacatcaccatgatgaagggcgtggacgactgcttcgtgtacaagatccgcttcgacggcgtgaacttccccgccaacggccccgtgatgcagcgcaagaccctgaagtgggagcccagcaccgagaagatgtacgtgcgcgacggcgtgctgaagggcgacgtgaacatggccctgctgctggagggcggcggccactaccgctgcgacttcaagaccacctacaaggccaagaaggtggtgcagctgcccgactaccacttcgtggaccaccgcatcgagatcgtgagccacgacaaggactacaacaaggtgaagctgtacgagcacgccgaggcccacagcggcctgccccgccaggccaagtaaaggcttaatgaaaagccaaga

SEQ ID NO: 4 - protein, 227 aa, Artificial
  synthetic
MSVIKPDMKIKLRMEGAVNGHKFVIEGDGKGKPFEGKQTMDLTVIEGAPLPFAYDILTTVFDYGNRVFAKYPKDIPDYFKQTFPEGYSWERSMTYEDQGICIATNDITMMKGVDDCFVYKIRFDGVNFPANGPVMQRKTLKWEPSTEKMYVRDGVLKGDVNMALLLEGGGHYRCDFKTTYKAKKVVQLPDYHFVDHRIEIVSHDKDYNKVKLYEHAEAHSGLPRQAK

SEQ ID NO: 5 - DNA, 726 bp, Artificial
  synthetic
tcgacccctaaggaggccaccatgagcgtgatcaagcccgacatgaagatcaagctgcggatggagggcgccgtgaacggccacaagttcgtgatcgagggcgacgggaaaggcaagcccttcgagggcaagcagaccatggacctgaccgtgatcgagggcgcccccctgcccttcgcttatgacattctcaccaccgtgttcgactacggcaaccgtgtcttcgccaagtaccccaaggacatccccgactacttcaagcagaccttccccgagggctacagctgggagcgcagcatgacctacgaggaccagggcatctgcatcgctacaaacgacatcaccatgatgaagggcgtggacgactgcttcgtgtacaagatccgcttcgacggtgtgaacttccctgccaacggcccggttatgcagcgcaagaccctaaagtgggagcccagcaccgagaagatgtacgtgcgcgacggcgtactgaagggcgacgtgaacatggccctgctcttggagggcggcggccactaccgctgcgacttcaagaccacctacaaggccaagaaggtggtgcagctgcccgactaccacttcgtggaccaccgcatcgagatcgtgagccacgacaaggactacaacaaggtgaagctgtacgagcacgccgaggcccacagcggcctgccccgccaggccaagtaaaggcttaatgaaaagccaaga

SEQ ID NO: 6 - protein, 227 aa, Artificial; identical to SEQ ID NO: 4
  synthetic

SEQ ID NO: 7 - DNA, 726 bp, Artificial
  synthetic
tcgacccctaaggaggccaccatgagcgtgatcaagcccgacatgaagatcaagctgcggatggagggcgccgtgaacggccacaaattcgtgatcgagggcgacgggaaaggcaagccctttgagggtaagcagaccatggacctgaccgtgatcgagggcgcccccctgcccttcgcttatgacattctcaccaccgtgttcgactacggtaaccgtgtcttcgccaagtaccccaaggacatccctgactacttcaagcagaccttccccgagggctacagctgggagcgaagcatgacatacgaggaccagggaatctgtatcgctacaaacgacatcaccatgatgaagggggtggacgactgcttcgtgtacaaaatccgcttcgacggtgtgaacttccctgctaatggcccggtgatgcagcgcaagaccctaaagtgggagcccagtaccgagaagatgtacgtgcgggacggcgtactgaagggcgatgtgaacatggccctgctcttggaggggggcggccactaccgctgcgacttcaagaccacctacaaagccaagaaggtggtgcagcttcccgactaccacttcgtggaccaccgcatcgagatcgtgagccacgacaaggactacaacaaagtcaagctgtacgagcacgccgaggcccacagcggactgccccgccaggccaagtaaaggcttaatgaaaagccaaga

SEQ ID NO: 8 - protein, 227 aa, Artificial; identical to SEQ ID NO: 4
  synthetic

SEQ ID NO: 9 - DNA, 726 bp, Artificial
  synthetic
tcgacccctaaggaggccaccatgagcgtgatcaagcccgacatgaagatcaagctgcggatggagggcgccgtgaacggccacaaattcgtgatcgagggcgacgggaaaggcaagccctttgagggtaagcagaccatggacctgaccgtgatcgagggcgcccccctgcccttcgcttatgacattctcaccaccgtgttcgactacggtaaccgtgtcttcgccaagtaccccaaggacatccctgactacttcaagcagaccttccccgagggctactcgtgggagcgaagcatgacatacgaggaccagggaatctgtatcgctacaaacgacatcaccatgatgaagggggtggacgactgcttcgtgtacaaaatccgcttcgacggtgtgaacttccctgctaatggcccggtgatgcagcgcaagaccctaaagtgggagcccagtaccgagaagatgtacgtgcgggacggcgtactgaagggcgatgttaacatggcactgctcttggaggggggcggccactaccgctgcgacttcaagaccacctacaaagccaagaaggtggtgcagcttcccgactaccacttcgtggaccaccgcatcgagatcgtgagccacgacaaggactacaacaaagtcaagctgtacgagcacgccgaagcccacagcggactaccccgccaggccaagtaaaggcttaatgaaaagccaaga

SEQ ID NO: 10 - protein, 227 aa, Artificial; identical to SEQ ID NO: 4
  synthetic

SEQ ID NO: 11 - DNA, 726 bp, Artificial
  synthetic
tcgacccctaaggaggccaccatgggcgtgatcaagcccgacatgaagatcaagctgcggatggagggcgccgtgaacggccacaaattcgtgatcgagggcgacgggaaaggcaagccctttgagggtaagcagaccatggacctgaccgtgatcgagggcgcccccctgcccttcgcttatgacattctcaccaccgtgttcgactacggtaaccgtgtcttcgccaagtaccccaaggacatccctgactacttcaagcagaccttccccgagggctactcgtgggagcgaagcatgacatacgaggaccagggaatctgtatcgctacaaacgacatcaccatgatgaagggggtggacgactgcttcgtgtacaaaatccgcttcgacggtgtgaacttccctgctaatggcccggtgatgcagcgcaagaccctaaagtgggagcccagtaccgagaagatgtacgtgcgggacggcgtactgaagggcgatgttaacatggcactgctcttggaggggggcggccactaccgctgcgacttcaagaccacctacaaagccaagaaggtggtgcagcttcccgactaccacttcgtggaccaccgcatcgagatcgtgagccacgacaaggactacaacaaagtcaagctgtacgagcacgccgaagcccacagcggactaccccgccaggccaagtaaaggcttaatgaaaagccaaga

SEQ ID NO: 12 - protein, 227 aa, Artificial
  synthetic
MGVIKPDMKIKLRMEGAVNGHKFVIEGDGKGKPFEGKQTMDLTVIEGAPLPFAYDILTTVFDYGNRVFAKYPKDIPDYFKQTFPEGYSWERSMTYEDQGICIATNDITMMKGVDDCFVYKIRFDGVNFPANGPVMQRKTLKWEPSTEKMYVRDGVLKGDVNMALLLEGGGHYRCDFKTTYKAKKVVQLPDYHFVDHRIEIVSHDKDYNKVKLYEHAEAHSGLPRQAK

SEQ ID NO: 13 - DNA, 726 bp, Artificial
  synthetic
tcgacccctaaggaggccaccatgggcgtgatcaagcccgacatgaagatcaagctgcggatggagggcgccgtgaacggccacaaattcgtgatcgagggcgacgggaaaggcaagccctttgagggtaagcagactatggacctgaccgtgatcgagggcgcccccctgcccttcgcttatgacattctcaccaccgtgttcgactacggtaaccgtgtcttcgccaagtaccccaaggacatccctgactacttcaagcagaccttccccgagggctactcgtgggagcgaagcatgacatacgaggaccagggaatctgtatcgctacaaacgacatcaccatgatgaagggggtggacgactgcttcgtgtacaaaatccgcttcgacggtgtgaacttccctgctaatggcccggtgatgcagcgcaagaccctaaagtgggagcccagtaccgagaagatgtacgtgcgggacggcgtactgaagggcgatgttaacatggcactgctcttggaggggggcggccactaccgctgcgacttcaagaccacctacaaagccaagaaggtggtgcagcttcccgactaccacttcgtggaccaccgcatcgagatcgtgagccacgacaaggactacaacaaagtcaagctgtacgagcacgccgaagcccacagcggactaccccgccaggccaagtaaaggcttaatgaaaagccaaga

SEQ ID NO: 14 - protein, 227 aa, Artificial; identical to SEQ ID NO: 12
  synthetic

SEQ ID NO: 15 - DNA, 746 bp, Artificial
  synthetic
nnnctcactataggctagcgatatccccgggggccaccatgggcgtgatcaagcccgacatgaagatcaagctgcggatggagggcgccgtgaacggccacaaattcgtgatcgagggcgacgggaaaggcaagccctttgagggtaagcagactatggacctgaccgtgatcgagggcgcccccctgcccttcgcttatgacattctcaccaccgtgttcgactacggtaaccgtgtcttcgccaagtaccccaaggacatccctgactacttcaagcagaccttccccgagggctactcgtgggagcgaagcatgacatacgaggaccagggaatctgtatcgctacaaacgacatcaccatgatgaagggggtggacgactgcttcgtgtacaaaatccgcttcgacggtgtgaacttccctgctaatggcccggtgatgcagcgcaagaccctaaagtgggagcccagtaccgagaagatgtacgtgcgggacggcgtactgaagggcgatgttaacatggcactgctcttggaggggggcggccactaccgctgcgacttcaagaccacctacaaagccaagaaggtggtgcagcttcccgactaccacttcgtggaccaccgcatcgagatcgtgagccacgacaaggactacaacaaagtcaagctgtacgagcacgccgaagcccacagcggactaccccgccaggccggctaattctagagcggccgcttcgagnnn

SEQ ID NO: 16 - protein, 227 aa, Artificial; identical to SEQ ID NO: 2
  synthetic

SEQ ID NO: 17 - DNA, 745 bp, Artificial
  synthetic
nnnctcactataggctagcgatatccccggggccaccatgggcgtgatcaagcccgacatgaagatcaagctgcggatggagggcgccgtgaacggccacaaattcgtgatcgagggcgacgggaaaggcaagccctttgagggtaagcagactatggacctgaccgtgatcgagggcgcccccctgcccttcgcttatgacattctcaccaccgtgttcgactacggtaaccgtgtcttcgccaagtaccccaaggacatccctgactacttcaagcagaccttccccgagggctactcgtgggagcgaagcatgacatacgaggaccagggaatctgtatcgctacaaacgacatcaccatgatgaagggggtggacgactgcttcgtgtacaaaatccgcttcgacggtgtgaacttccctgctaatggcccggtgatgcagcgcaagaccctaaagtgggagcccagtaccgagaagatgtacgtgcgggacggcgtactgaagggcgatgttaacatggcactgctcttggaggggggcggccactaccgctgcgacttcaagaccacctacaaagccaagaaggtggtgcagcttcccgactaccacttcgtggaccaccgcatcgagatcgtgagccacgacaaggactacaacaaagtcaagctgtacgagcacgccgaagcccacagcggactaccccgccaggccggctaattctagagcggccgcttcgagnnn

SEQ ID NO: 18 - protein, 227 aa, Artificial; identical to SEQ ID NO: 2
  synthetic

SEQ ID NO: 19 - DNA, 748 bp, Artificial
  synthetic
nnnctcactataggctagccccggggatatcgccaccatgggcgtgatcaagcccgacatgaagatcaagctgcggatggagggcgccgtgaacggccacaaattcgtgatcgagggcgacgggaaaggcaagccctttgagggtaagcagactatggacctgaccgtgatcgagggcgcccccctgcccttcgcttatgacattctcaccaccgtgttcgactacggtaaccgtgtcttcgccaagtaccccaaggacatccctgactacttcaagcagaccttccccgagggctactcgtgggagcgaagcatgacatacgaggaccagggaatctgtatcgctacaaacgacatcaccatgatgaagggtgtggacgactgcttcgtgtacaaaatccgcttcgacggggtcaacttccctgctaatggcccggtgatgcagcgcaagaccctaaagtgggagcccagtaccgagaagatgtacgtgcgggacggcgtactgaagggcgatgttaatatggcactgctcttggagggaggcggccactaccgctgcgacttcaagaccacctacaaagccaagaaggtggtgcagcttcccgactaccacttcgtggaccaccgcatcgagatcgtgagccacgacaaggactacaacaaagtcaagctgtacgagcacgccgaagcccacagcggactaccccgccaggccggctaatagttctagagcggccgcttcgagnnn

SEQ ID NO: 20 - protein, 227 aa, Artificial; identical to SEQ ID NO: 2
  synthetic

SEQ ID NO: 21 - DNA, 684 bp, Artificial
  parent
atgagtgtgataaaaccagacatgaagatcaagctgcgtatggaaggtgctgtaaacgggcacaagttcgtgattgaaggagacggaaaaggcaagcctttcgagggaaaacagactatggaccttacagtcatagaaggcgcacctttgcctttcgcttacgatatcttgacaacagtattcgattacggcaacagggtattcgccaaatacccaaaagacataccagactatttcaagcagacgtttccggaggggtactcctgggaacgaagcatgacatacgaagaccagggcatttgcatcgccacaaacgacataacaatgatgaaaggcgtcgacgactgttttgtctataaaattcgatttgatggtgtgaactttcctgccaatggtccagttatgcagaggaagacgctaaaatgggagccatccactgaaaaaatgtatgtgcgtgatggggtactgaagggtgatgttaacatggctctgttgcttgaaggaggtggccattaccgatgtgacttcaaaactacttacaaagctaagaaggttgtccagttgccagactatcattttgttgaccatcgcattgagattgtgagccacgacaaagattacaacaaggttaagctgtatgagcatgccgaagctcattctgggctgccgaggcaggccaagtaa

SEQ ID NO: 22 - protein, 227 aa, Artificial; identical to SEQ ID NO: 4
  parent

(19) United States
(12) Patent Application Publication
(10) Pub. No.: US 2003/0157643 A1
(43) Pub. Date: Aug. 21, 2003

(54) Synthetic nucleic acids from aquatic species

(76) Inventors: Brian D. Almond, Fitchburg, WI (US); Monika G. Wood, Mt. Horeb, WI (US); Keith V. Wood, Mt. Horeb, WI (US)

Correspondence Address: Mary E. Eberle, BOYLE, FREDRICKSON, NEWHOLM, STEIN & GRATZ, S.C., 250 Plaza, Suite 1030, 250 East Wisconsin Avenue, Milwaukee, WI, 53202, US

(21) Appl. No.: 10/314,827

(22) Filed: Dec. 9, 2002

Related U.S. Application Data
(63) Continuation-in-part of application No. 09/645,706, filed on Aug. 24, 2000.

(51) Int. Cl. C07H 21/04; C12P 21/02; C07K 14/435; C12N 5/06

(52) U.S. Cl. 435/69.1; 435/320.1; 435/325; 530/350; 536/23.2

(57) ABSTRACT

A synthetic nucleic acid molecule is provided that includes nucleotides of a coding region for a fluorescent polypeptide having a codon composition differing at more than 25% of the codons from a parent nucleic acid sequence encoding a fluorescent polypeptide. The synthetic nucleic acid molecule has at least 3-fold fewer transcription regulatory sequences relative to the average number of such sequences in the parent nucleic acid sequence. The polypeptide encoded by the synthetic nucleic acid molecule preferably has at least 85% sequence identity to the polypeptide encoded by the parent nucleic acid sequence.

### CROSS-REFERENCE TO RELATED APPLICATIONS

This application claims priority under 35 U.S.C. §120 to U.S. patent application Ser. No. 09/645,706, filed Aug. 24, 2000, the entirety of which is incorporated by reference herein.

### BIBLIOGRAPHY

Complete bibliographic citations of the references referred to herein by the first author's last name in parentheses can be found in the Bibliography section, immediately preceding the claims.

### FIELD OF THE INVENTION

The invention relates to the field of biochemical assays and reagents. More specifically, this invention relates to fluorescent proteins and to methods for their use.

### BACKGROUND OF THE INVENTION

Transcription, the synthesis of an RNA molecule from a sequence of DNA is the first step in gene expression. Genetic elements that regulate DNA transcription include promoters, polyadenylation signals, transcription factor binding sites and enhancers. A promoter is capable of specific initiation of transcription and typically is composed of three general regions. The core promoter is where the RNA polymerase and its cofactors bind to the DNA. Immediately upstream of the core promoter is the proximal promoter, which contains several transcription factor binding sites that are responsible for the assembly of an activation complex that in turn recruits the polymerase complex. The distal promoter, located further upstream of the proximal promoter also contains transcription factor binding sites. Transcription termination and polyadenylation, like transcription initiation, are specific genetic elements. Enhancers typically contain multiple transcription factor binding sites that can significantly increase the level of transcription from a responsive promoter regardless of the enhancer's orientation and distance with respect to the promoter as long as the enhancer and promoter are located within the same DNA molecule. The amount of transcript produced from a gene may also be regulated by a post-transcriptional mechanism, the most important being RNA splicing that removes intervening sequences (introns) from a primary transcript between the splice donor and splice acceptor. Genetic elements located within a DNA molecule, including promoters, enhancers, polyadenylation sites, transcription factor binding sites, and RNA splice sites, are typically correlatable with recognizable sequences. These sequences are generally believed to be an essential component to the functioning of a genetic element. Thus, for example, a promoter sequence is a specific sequence or group of sequences that has been found to correlate with, promoter function.

Natural selection is the hypothesis that genotype-environment interactions occurring at the phenotypic level lead to differential reproductive success of individuals and therefore to modification of the gene pool of a population. Some properties of nucleic acid molecules that are acted upon by natural selection include codon usage frequency, RNA secondary structure, the efficiency of intron splicing, and interactions with transcription factors or other nucleic acid binding proteins. Because of the degenerate nature of the genetic code, mutations within the coding regions of genes can occur through natural selection to optimize these properties without altering the corresponding amino acid sequence.

Under some conditions, it is useful to synthetically alter the natural nucleotide sequence encoding a polypeptide to better adapt the polypeptide for alternative applications. A common example is to alter the codon usage frequency of a gene when it is expressed in a foreign host cell. Although redundancy in the genetic code allows amino acids to be encoded by multiple codons, different organisms favor some codons over others. It has been found that the efficiency of protein translation in a non-native host cell can be substantially increased by adjusting the codon usage frequency but maintaining the same gene product (U.S. Pat. Nos. 5,096,825, 5,670,356, and 5,874,304).

However, altering codon usage may, in turn, result in the unintentional introduction into a synthetic nucleic acid molecule of inappropriate transcription regulatory sequences. This may adversely effect transcription, resulting in anomalous expression of the synthetic DNA. Anomalous expression is defined as departure from normal or expected levels of expression. For example, transcription factor binding sites located downstream from a promoter have been demonstrated to effect promoter activity (Michael et al., 1990; Lamb et al., 1998; Johnson et al., 1998; Jones et al., 1997). Additionally, it is not uncommon for an enhancer sequence to exert activity and result in elevated levels of DNA transcription in the absence of a promoter or for the presence of transcription regulatory sequences to increase the basal levels of gene expression in the absence of a promoter.

Fluorescent proteins are proteins that fluoresce when excited by light. Fluorescent proteins can be used in a number of assays and diagnostic procedures and to study gene expression and protein localization. A problem with existing fluorescent proteins occurs when they are expressed in species that are genetically distant from which they have been isolated. In this situation, they are typically expressed at low levels, making detection of the fluorescent proteins difficult. One of the reasons for this may be codon preference. For instance, plant genes tend to use certain codons over other codons. In addition, within plants, highly expressed genes have particular codon preferences. (Wada et al., 1990, Murray et al., 1989). Animal genes also show codon preferences. For example, humans also show codon preferences.

Thus, what is needed are synthetic nucleic acid molecules that encode fluorescent proteins and that have codon compositions differing from a parent nucleic acid sequences encoding fluorescent polypeptides. Preferably, the synthetic nucleic acid molecules with altered codon usage do not have inappropriate or unintended transcription regulatory sequences for expression in a particular host cell. This would permit higher levels of expression in a host cell that differs from the source from which the fluorescent protein was originally isolated. Moreover, fluorescent proteins having higher levels of expression permit improved detection of the fluorescent proteins.

### SUMMARY OF THE INVENTION

The invention, which is defined by the claims set out at the end of this disclosure, is intended to solve at least some of the problems noted above. The invention provides a synthetic nucleic acid molecule comprising nucleotides of a coding region for a fluorescent polypeptide having a codon composition differing at more than 25% of the codons from a parent nucleic acid sequence encoding a fluorescent polypeptide and having at least 3-fold fewer transcription regulatory sequences relative to the average number of such sequences in the parent nucleic acid sequence. Preferably, the synthetic nucleic acid molecule encodes a polypeptide that has an amino acid sequence that is at least 85%, preferably at least 90%, and most preferably at least 95% or at least 99% identical to the amino acid sequence of the parent (parent or another synthetic) polypeptide (protein) from which it is derived. Thus, it is recognized that some specific amino acid changes may also be desirable to alter a particular phenotypic characteristic of the polypeptide encoded by the synthetic nucleic acid molecule. Preferably, the amino acid sequence identity is over at least 100 contiguous amino acid residues. In one embodiment of the invention, the codons in the synthetic nucleic acid molecule that differ preferably encode the same amino acids as the corresponding codons in the parent nucleic acid sequence.

The transcription regulatory sequences that are reduced in the synthetic nucleic acid molecule include, but are not limited to, any combination of transcription factor binding sequences, intron splice sequences, poly(A) addition sequences, enhancer sequences and promoter sequences. Transcription regulatory sequences are well known in the art. It is preferred that the synthetic nucleic acid molecule of the invention has a codon composition that differs from that of the parent nucleic acid sequence at more than 25%, 30%, 35%, 40% or more than 45%, e.g., 50%, 55%, 60% or more of the codons. Codons for use in the invention are those which are employed more frequently than at least one other codon for the same amino acid in a particular organism and, more preferably, are also not low-usage codons in that organism and are not low-usage codons in the organism, for example, E. coli, used to clone or screen for the expression of the synthetic nucleic acid molecule. Moreover, preferred codons for certain amino acids, i.e., those amino acids that have three or more codons, may include two or more codons that are employed more frequently than the other (non-preferred) codon(s). The presence of codons in the synthetic nucleic acid molecule that are employed more frequently in one organism than in another organism results in a synthetic nucleic acid molecule which, when introduced into the cells of the organism that employs those codons more frequently, is expressed in those cells at a level that is greater than the expression of the parent nucleic acid sequence in those cells. For example, the synthetic nucleic acid molecule of the invention is expressed at a level that is at least about 105%, e.g., 110%, 150%, 200%, 500% or more (e.g., 1000%, 5000%, or 10000%), of that of the parent nucleic acid sequence in a cell or cell extract under identical conditions (such as cell culture conditions, vector backbone, and the like).

In one embodiment of the invention, the codons that are different are those employed more frequently in a mammal, while in another embodiment the codons that are different are those employed more frequently in a plant. A particular type of mammal, e.g., human, may have a different set of preferred codons than another type of mammal. Likewise, a particular type of plant may have a different set of preferred codons than another type of plant. In addition, certain other types of factors, such as highly expressed genes within plants or animals, may have a different set of preferred codons than lowly expressed genes. In one embodiment of the invention, the majority of the codons that differ are ones that are preferred codons in a desired host cell. Preferred codons for mammals (e.g., humans) and plants are known to the art (e.g., Wada et al., 1990). For example, preferred human codons include, but are not limited to, CGC (Arg), CTG (Leu), TCT (Ser), AGC (Ser), ACC (Thr), CCA (Pro), CCT (Pro), GCC (Ala), GGC (Gly), GTG (Val), ATC (Ile), ATT (Ile), AAG (Lys), AAC (Asn), CAG (Gln), CAC (His), GAG (Glu), GAC (Asp), TAC (Tyr), TGC (Cys) and TTC (Phe) (Wada et al., 1990). Thus, preferred “humanized” synthetic nucleic acid molecules of the invention have a codon composition which differs from a parent nucleic acid sequence by having an increased number of the preferred human codons, e.g. CGC, CTG, TCT, AGC, ACC, CCA, CCT, GCC, GGC, GTG, ATC, ATT, AAG, AAC, CAG, CAC, GAG, GAC, TAC, TGC, TTC, or any combination thereof. For example, the synthetic nucleic acid molecule of the invention may have an increased number of CTG or TTG leucine-encoding codons, GTG or GTC valine-encoding codons, GGC or GGT glycine-encoding codons, ATC or ATT isoleucine-encoding codons, CCA or CCT proline-encoding codons, CGC or CGT arginine-encoding codons, AGC or TCT serine-encoding codons, ACC or ACT threonine-encoding codon, GCC or GCT alanine-encoding codons, or any combination thereof, relative to the parent nucleic acid sequence.

Similarly, synthetic nucleic acid molecules having an increased number of codons that are employed more frequently in plants, have a codon composition which differs from a parent nucleic acid sequence by having an increased number of the plant codons including, but not limited to, CGC (Arg), CTT (Leu), TCT (Ser), TCC (Ser), ACC (Thr), CCA (Pro), CCT (Pro), GCT (Ser), GGA (Gly), GTG (Val), ATC (Ile), ATT (Ile), AAG (Lys), AAC (Asn), CAA (Gln), CAC (His), GAG (Glu), GAC (Asp), TAC (Tyr), TGC (Cys), TTC (Phe), or any combination thereof (Murray et al., 1989). Preferred codons may differ for different types of plants (Wada et al., 1990).

The choice of codon may be influenced by many factors such as, for example, the desire to have an increased number of nucleotide substitutions or decreased number of transcription regulatory sequences. Under some circumstances, e.g., to perrnit removal of a transcription factor binding sequence, it may be desirable to replace a non-preferred codon with a codon other than a preferred codon or a codon other than the most preferred codon. Under other circumstances, for example, to prepare codon distinct versions of a synthetic nucleic acid molecule, preferred codon pairs are selected based upon the largest number of mismatched bases, as well as the criteria described above.

The presence of codons in the synthetic nucleic acid molecule that are employed more frequently in one organism than in another organism, results in a synthetic nucleic acid molecule which, when introduced into a cell of the organism that employs those codons, is expressed in that cell at a level which is greater than the level of expression of the parent nucleic acid sequence.

In one embodiment of a synthetic nucleic acid molecule of the invention that is a fluorescent protein, the synthetic nucleic acid molecule encodes a green fluorescent protein having a codon composition different than that of a parent green fluorescent protein nucleic acid sequence. A synthetic green fluorescent protein nucleic acid molecule of the invention may optionally encode the amino acid glycine at position 2, or may optionally encode the amino acid glycine at position 227 or a combination of the amino acid glycine at position 2 and the amino acid glycine at position 227. Preferred synthetic green fluorescent protein nucleic acid molecules include, but are not limited to, those derived from Montastraea cavernosa.

The invention also provides a vector construct. The vector construct of the invention comprises a synthetic vector backbone having at least 3-fold fewer transcriptional regulatory sequences relative to a parent vector backbone. The vector construct also comprises a nucleic acid molecule comprising nucleotides of a coding region for a fluorescent polypeptide having a codon composition differing at more than 25% of the codons from a parent nucleic acid sequence encoding a fluorescent polypeptide and having at least 3-fold fewer transcription regulatory sequences relative to the average number of such sequences in the parent nucleic acid sequence.

A plasmid is additionally provided. The plasmid comprises a nucleic acid molecule comprising nucleotides of a coding region for a fluorescent polypeptide having a codon composition differing at more than 25% of the codons from a parent nucleic acid sequence encoding a fluorescent polypeptide and having at least 3-fold fewer transcription regulatory sequences relative to the average number of such sequences in the parent nucleic acid sequence.

In addition, an expression vector is provided. The expression vector comprises a nucleic acid molecule comprising nucleotides of a coding region for a fluorescent polypeptide having a codon composition differing at more than 25% of the codons from a parent nucleic acid sequence encoding a fluorescent polypeptide and having at least 3-fold fewer transcription regulatory sequences relative to the average number of such sequences in the parent nucleic acid sequence. The nucleic acid molecule is linked to a promoter functional in a cell.

Also provided is a host cell comprising the expression vector and kits comprising the expression vector in a suitable container.

The invention also provides a method to prepare a synthetic nucleic acid molecule of the invention by genetically altering a parent (either wild type or another synthetic) nucleic acid sequence. The method may be used to prepare a synthetic nucleic acid molecule encoding a fluorescent protein. The method of the invention may be employed to alter the codon usage frequency and decrease the number of transcription regulatory sequences in an open reading frame of any protein (e.g., a fluorescent protein) or to decrease the number of transcription regulatory sites in a vector backbone. Preferably, the codon usage frequency in the synthetic nucleic acid molecule is altered to reflect that of the host organism desired for expression of that nucleic acid molecule while also decreasing the number of potential transcription regulatory sequences relative to the parent nucleic acid molecule.

Thus, the invention provides a method to prepare a synthetic nucleic acid molecule comprising an open reading frame. The method comprises altering a plurality of transcription regulatory sequences in a parent nucleic acid sequence which encodes a fluorescent polypeptide to yield a synthetic nucleic acid molecule which has at least 3-fold fewer transcription regulatory sequences relative to the parent nucleic acid sequence. The method also comprises altering greater than 25% of the codons in the synthetic nucleic acid sequence which has a decreased number of transcription regulatory sequences to yield a further synthetic nucleic acid molecule. The codons which are altered do not result in an increased number of transcription regulatory sequences. The further synthetic nucleic acid molecule encodes a polypeptide with at least 85% amino acid sequence identity to the polypeptide encoded by the parent nucleic acid sequence.

Alternatively, the method comprises altering greater than 25% of the codons in a parent nucleic acid sequence which encodes a fluorescent polypeptide to yield a codon-altered synthetic nucleic acid molecule. The method also comprises altering a plurality of transcription regulatory sequences in the codon-altered synthetic nucleic acid molecule to yield a further synthetic nucleic acid molecule which has at least 3-fold fewer transcription regulatory sequences relative to a synthetic nucleic acid molecule with codons which differ from the corresponding codons in the parent nucleic acid sequence. The further synthetic nucleic acid molecule encodes a polypeptide with at least 85% amino acid sequence identity to the fluorescent polypeptide encoded by the parent nucleic acid sequence.

As described hereinbelow, the methods of the invention were employed with Montastraea cavernosa green fluorescent protein (McGFP) nucleic acid sequences to generate a synthetic nucleic acid that is more readily expressed in human cells. Disclosed herein are synthetic nucleic acid molecule sequences that encode highly related polypeptides. These synthetic nucleic acid molecules include intermediates in the method of the invention and hGreen II. These synthetic nucleic acid molecules have a number of nucleotide differences relative to each other.

The method of the invention produced a synthetic nucleic acid molecule which exhibited significantly enhanced levels of mammalian expression without negatively effecting other desirable physical or biochemical properties (including protein half-life) and which had a greatly reduced number of known transcription regulatory sequences.

The invention also provides at least two synthetic nucleic acid molecules that encode highly related polypeptides, but which synthetic nucleic acid molecules have an increased number of nucleotide differences relative to each other. These differences decrease the recombination frequency between the two synthetic nucleic acid molecules when those molecules are both present in a cell (i.e., they are “codon distinct” versions of a synthetic nucleic acid molecule). Thus, the invention provides a method for preparing at least two synthetic nucleic acid molecules that are codon distinct versions of a parent nucleic acid sequence that encodes a polypeptide. The method comprises altering a parent nucleic acid sequence to yield a first synthetic nucleic acid molecule having an increased number of a first plurality of codons that are employed more frequently in a selected host cell relative to the number of those codons present in the parent nucleic acid sequence. Optionally, the first synthetic nucleic acid molecule also has a decreased number of transcription regulatory sequences relative to the parent nucleic acid sequence. The parent nucleic acid sequence is also altered to yield a second synthetic nucleic acid molecule having an increased number of a second plurality of codons that are employed more frequently in the host cell relative to the number of those codons in the parent nucleic acid sequence. The first plurality of codons is different than the second plurality of codons. The first and the second synthetic nucleic acid molecules preferably encode the same polypeptide. Optionally, the second synthetic nucleic acid molecule has a decreased number of transcription regulatory sequences relative to the parent nucleic acid sequence. Either or both synthetic molecules can then be further modified.

### BRIEF DESCRIPTION OF THE DRAWINGS

Preferred exemplary embodiments of the invention are illustrated in the accompanying drawings in which:

FIG. 1 shows codons and their corresponding amino acids.

FIGS. 2A-2B show a sequence alignment of the DNA sequence (SEQ. ID. NO:1) encoding a humanized green fluorescent protein and the DNA sequence (SEQ. ID. NO:21) encoding a protein (Green II)) derived from a Montastraea cavernosa protein. The humanized hGreen II was generated from Green II. In this alignment, the differences between the sequences being aligned are indicated by a missing monomer in the “consensus” line.

FIG. 3 shows an amino acid alignment of the amino acids encoded by the DNA sequences of hGreen II (SEQ. ID. NO:2) and Green II (SEQ. ID. NO:22). In this alignment, the differences between the sequences being aligned are indicated by a missing monomer in the “consensus” line.

FIGS. 4A-4D show a sequence alignment of the DNA encoding intermediates between Green II and hGreen II, described in Example 1 below. In this alignment, lower case letters denote the flanking sequences and upper case letter the gene coding regions.

FIGS. 5A-5B are graphs showing transfection efficiency (top/large rectangle) and log of fluorescence of 50,000 CHO cells transfected with a Green II vector construct (FIG. 5A) and a hGreen II vector construct (FIG. 5B) assayed by FACS twenty-four hours after transfection.

FIGS. 6A-6B are graphs showing transfection efficiency (top/large rectangle) and log of fluorescence of 50,000 CHO cells transfected with a Green II vector construct (FIG. 6A) and a hGreen II vector construct (FIG. 6B) assayed by FACS twenty-four hours after transfection.

FIGS. 7A-7B are graphs showing transfection efficiency (top/large rectangle) and log of fluorescence of 50,000 NIH 3T3 cells transfected with a Green II vector construct (FIG. 7A) and a hGreen II vector construct (FIG. 7B) assayed by FACS twenty-four hours after transfection.

FIGS. 8A-8F show images of NIH 3T3 cells that were transfected with a Green II vector construct and a hGreen II vector construct at 2, 3, and 6 days.

FIG. 9 is a graph showing NIH 3T3 cells transfected with a luciferase reporter plus increasing concentrations of a Green II vector construct and an hGreen II vector construct. Firefly luciferase was used as a reporter of cytoxicity.

Before explaining embodiments of the invention in detail, it is to be understood that the invention is not limited in its application to the details of construction and the arrangement of the components set forth in the following description or illustrated in the drawings. The invention is capable of other embodiments or being practiced or carried out in various ways. Also, it is to be understood that the phraseology and terminology employed herein is for the purpose of description and should not be regarded as limiting.

### DETAILED DESCRIPTION

Definitions:

For purposes of the present invention, the following definitions apply:

The term “gene” as used herein, refers to a DNA sequence that comprises coding sequences necessary for the production of a polypeptide or protein precursor. The polypeptide can be encoded by a full-length coding sequence or by any portion of the coding sequence, as long as the desired protein activity is retained.

As used herein, “amino acids” are described in keeping with standard polypeptide nomenclature, J. Biol. Chem., 243:3557-59, (1969).

The standard, one-letter codes “A,” “C,” “G,” “T,” “U,” and “I” are used herein for the nucleotides adenine, cytosine, guanine, thymine, uracil, and inosine, respectively. “N” designates any nucleotide. Oligonucleotide or polynucleotide sequences are written from the 5′-end to the 3′-end.

All amino acid residues identified herein are in the natural L-configuration. In keeping with standard polypeptide nomenclature, abbreviations for amino acid residues are as shown in the following Table of Correspondence.

TABLE OF CORRESPONDENCE

| 1-Letter | 3-Letter | AMINO ACID |
|---|---|---|
| Y | Tyr | L-tyrosine |
| G | Gly | glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| W | Trp | L-tryptophan |
| R | Arg | L-arginine |
| D | Asp | L-aspartic acid |
| N | Asn | L-asparagine |
| C | Cys | L-cysteine |

The term “isolated” when used in relation to a nucleic acid, as in “isolated nucleic acid” or “isolated polynucleotide,” refers to a nucleic acid sequence that is identified and separated from at least one contaminant with which it is ordinarily associated in its source. Thus, an isolated nucleic acid is present in a form or setting that is different from that in which it is found in nature. In contrast, non-isolated nucleic acids, e.g., DNA and RNA, are found in the state they exist in nature. For example, a given DNA sequence, e.g., a gene, is found on the host cell chromosome in proximity to neighboring genes; RNA sequences, e.g., a specific mRNA sequence encoding a specific protein, are found in the cell as a mixture with numerous other mRNAs that encode a multitude of proteins. However, isolated nucleic acid includes, by way of example, such nucleic acid in cells ordinarily expressing that nucleic acid where the nucleic acid is in a chromosomal location different from that of natural cells, or is otherwise flanked by a different nucleic acid sequence than that found in nature. The isolated nucleic acid may be present in single-stranded or double-stranded form. When an isolated nucleic acid is to be utilized to express a protein, the oligonucleotide contains at a minimum, the sense or coding strand, i.e., the oligonucleotide may single-stranded, but may contain both the sense and anti-sense strands, i.e., the oligonucleotide may be double-stranded.

The term “isolated” when used in relation to a polypeptide, as in “isolated protein” or “isolated polypeptide” refers to a polypeptide that is identified and separated from at least one contaminant with which it is ordinarily associated in its source. Thus, an isolated polypeptide is present in a form or setting that is different from that in which it is found in nature. In contrast, non-isolated polypeptides, e.g., proteins and enzymes, are found in the state they exist in nature.

The term “purified” or “to purify” means the result of any process that removes some of a contaminant from the component of interest, such as a protein or nucleic acid. The percent of a purified component is thereby increased in the sample.

With reference to nucleic acids of the invention, the term “nucleic acid” refers to DNA, genomic DNA, cDNA, RNA, mRNA and a hybrid of the various nucleic acids listed. The nucleic acid can be of synthetic origin or natural origin. A nucleic acid, as used herein, is a covalently linked sequence of nucleotides in which the 3′ position of the pentose of one nucleotide is joined by a phosphodiester group to the 5′ position of the pentose of the next, and in which the nucleotide residues (bases) are linked in specific sequence, i.e., a linear order of nucleotides. A “polynucleotide,” as used herein, is a nucleic acid containing a sequence that is greater than about 100 nucleotides in length. An “oligonucleotide,” as used herein, is a short polynucleotide or a portion of a polynucleotide. An oligonucleotide typically contains a sequence of about two to about one hundred bases. The word “oligo” is sometimes used in place of the word “oligonucleotide.”

Nucleic acid molecules are said to have a “5′-terminus” (5′ end) and a “3′-terminus” (3′ end) because nucleic acid phosphodiester linkages occur to the 5′ carbon and 3′ carbon of the pentose ring of the substituent mononucleotides. The end of a polynucleotide at which a new linkage would be to a 5′ carbon is its 5′ terminal nucleotide. The end of a polynucleotide at which a new linkage would be to a 3′ carbon is its 3′ terminal nucleotide. A terminal nucleotide, as used herein, is the nucleotide at the end position of the 3′- or 5′-terminus.

As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. Typically, promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

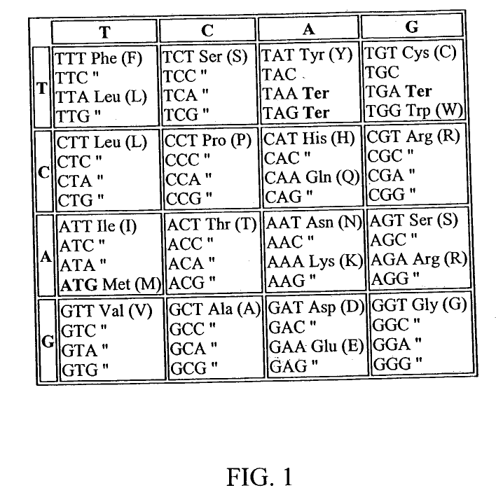

The term “codon” as used herein, is a basic genetic coding unit, consisting of a sequence of three nucleotides that specify a particular amino acid to be incorporated into a polypeptide chain, or a start or stop signal. FIG. 1 contains a codon table. The term “coding region” when used in reference to a structural gene refers to the nucleotide sequences that encode the amino acids found in the polypeptide as a result of translation of a mRNA molecule. Typically, the coding region is bounded on the 5′ side by the nucleotide triplet “ATG” which encodes the initiator methionine and on the 3′ side by a stop codon (e.g., TAA, TAG, TGA). In some cases the coding region is also known to initiate by a nucleotide triplet “TTG.”

By “protein” and “polypeptide” is meant any chain of amino acids, regardless of length or post-translational modification, e.g., glycosylation or phosphorylation. The synthetic genes of the invention may also encode a variant of a parent protein or polypeptide fragment thereof. Preferably, such a protein polypeptide has an amino acid sequence that is at least 85%, preferably at least 90%, and most preferably at least 95% or at least 99% identical to the amino acid sequence of the parent protein or polypeptide from which it is derived.

Polypeptide molecules are said to have an “amino terminus” (N-terminus) and a “carboxy terminus” (C-terminus) because peptide linkages occur between the backbone amino group of a first amino acid residue and the backbone carboxyl group of a second amino acid residue. The terms “N-terminal” and “C-terminal” in reference to polypeptide sequences refer to regions of polypeptides including portions of the N-terminal and C-terminal regions of the polypeptide, respectively. A sequence that includes a portion of the N-terminal region of polypeptide includes amino acids predominantly from the N-terminal half of the polypeptide chain, but is not limited to such sequences. For example, an N-terminal sequence may include an interior portion of the polypeptide sequence including bases from both the N-terminal and C-terminal halves of the polypeptide. The same applies to C-terminal regions. N-terminal and C-terminal regions may, but need not, include the amino acid defining the ultimate N-terminus and C-terminus of the polypeptide, respectively.

The term “wild type” as used herein, refers to a gene or gene product that has the characteristics of that gene or gene product isolated from a naturally occurring source. A wild type gene is that which is most frequently observed in a native population and is thus arbitrarily designated the wild type form of the gene. In contrast, the term “mutant” refers to a gene or gene product that displays modifications in sequence and/or functional properties, i.e., altered characteristics, when compared to the wild type gene or gene product. It is noted that naturally-occurring mutants can be isolated; these are identified by the fact that they have altered characteristics when compared to the wild type gene or gene product.

The terms “complementary” or “complementarity” are used in reference to a sequence of nucleotides related by the base-pairing rules. For example, for the sequence 5′“A-G-T′3′, is complementary to the sequence 3′“T-C-A” 5′. Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon hybridization of nucleic acids.

The term “recombinant protein” or “recombinant polypeptide” as used herein refers to a protein molecule expressed from a recombinant DNA molecule. In contrast, the term “native protein” is used herein to indicate a protein isolated from a naturally occurring (i.e., a nonrecombinant) source. Molecular biological techniques may be used to produce a recombinant form of a protein with identical properties as compared to the native form of the protein.

The terms “fusion protein” and “fusion partner” refer to a chimeric protein containing a protein of interest, e.g., a fluorescent protein, joined to an exogenous protein fragment, e.g., a fusion partner that consists of a second protein, (e.g., a fluorescent or non-fluorescent protein or a peptide). The fusion partner may enhance the solubility of protein as expressed in a host cell, may, for example, provide an affinity tag to allow purification of the recombinant fusion protein from the host cell or culture supernatant, or both. If desired, the fusion partner may be removed from the protein of interest by a variety of enzymatic or chemical means known to the art. In addition, the exogenous protein fragment may be another protein of interest that is fused to the fluorescent protein. This permits the tracking of the exogenous protein fragment with fluorescence.

The term “nucleic acid construct” denotes a nucleic acid that is composed of two or more distinct or discreet nucleic acid sequences and that are ligated together or synthesized using methods known in the art.

The term “parent” refers to a naturally occurring or non-naturally occurring nucleic acid or protein. Parent is used to denote the material from which a synthetic nucleic acid or synthetic protein is generated.

The terms “cell,” “cell line,” “host cell,” as used herein, are used interchangeably, and all such designations include progeny or potential progeny of these designations. By “transformed cell” is meant a cell into which (or into an ancestor of which) has been introduced a DNA molecule. Optionally, a synthetic gene of the invention may be introduced into a suitable cell line so as to create a transfected (“stably” or “transient”) cell line capable of producing the protein or polypeptide encoded by the synthetic gene. Vectors, cells, and methods for constructing such cell lines are well known in the art, e.g. in Ausubel, et al (1992). The words “transformants” or “transformed cells” include the primary transformed cells derived from the originally transformed cell without regard to the number of transfers. All progeny may not be precisely identical in DNA content, due to deliberate or inadvertent mutations. Nonetheless, mutant progeny that have the same functionality as screened for in the originally transformed cell are included in the definition of transformants.

Nucleic acids are known to contain different types of mutations. A “point” mutation refers to an alteration in the sequence of a nucleotide at a single base position from the wild type or parent sequence. Mutations may also refer to insertion or deletion of one or more bases, so that the nucleic acid sequence differs from the wild type or parent sequence.

The term “operably linked” as used herein refers to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional, e.g., enzymatically active, capable of binding to a binding partner, capable of inhibiting, protein or polypeptide, is produced.

The term “recombinant DNA molecule” means a hybrid DNA sequence comprising at least two nucleotide sequences not normally found together in nature.

The term “vector” is used in reference to a nucleic acid molecules into which fragments of DNA may be inserted or cloned and can be used to transfer nucleic acid segment(s) into a cell and is capable of replication in a cell. Vectors may be derived from plasmids, bacteriophages, viruses, cosmids, and the like, or generated synthetically.

The term “expression vector” as used herein refers to a vector containing appropriate DNA or RNA sequences necessary for the expression of an operably linked coding sequence in a particular host organism. Prokaryotic expression vectors typically include a promoter, a ribosome binding site, an origin of replication for autonomous replication in a host cell and possibly other elements, e.g. an optional operator, optional restriction enzyme sites.

The term “promoter” refers to a genetic element that directs RNA polymerase to bind to DNA and to initiate RNA synthesis. Eukaryotic expression vectors typically include a promoter, optionally a polyadenlyation signal, and optionally an enhancer.

The term “a polynucleotide having a nucleotide sequence encoding a gene,” means a nucleic acid sequence comprising the coding region of a gene, or in other words the nucleic acid sequence which encodes a gene product. The coding region may be present in either a cDNA, genomic DNA, or RNA form. When present in a DNA form, the oligonucleotide may be single-stranded or double-stranded. Suitable control elements, such as enhancers/promoters, splice junctions, polyadenylation signals, may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening regions, polyadenylation signals, etc. In further embodiments, the coding region may contain a combination of both endogenous and exogenous control elements.

The term “transcription regulatory element” refers to a genetic element that controls some aspect of the expression of nucleic acid sequence(s). For example, a promoter is a regulatory element that facilitates the initiation of transcription of an operably linked coding region. Other regulatory elements include, but are not limited to, transcription factor binding sites, splicing signals, polyadenylation signals, termination signals, and enhancer elements.

The term “transcription regulatory sequence” refers to nucleic acid sequences associated with the function of a transcription regulatory element. Such sequences are typically recognizable as sequence motifs, or corresponding to known consensus sequences, and are generally believed to be necessary for the function of the transcription regulatory element.

Transcriptional control signals in eukaryotes comprise “promoter” and “enhancer” elements. Promoters and enhancers typically comprise short arrays of DNA sequences that interact specifically with cellular proteins involved in transcription (Maniatis et al., 1987). Promoter and enhancer elements have been isolated from a variety of eukaryotic sources including genes in yeast, insect and mammalian cells. Promoter and enhancer elements have also been isolated from viruses and analogous control elements, such as promoters, are also found in prokaryotes. The function of a particular promoter and enhancer depends on the cell type used to express the protein of interest. Some eukaryotic promoters and enhancers have a broad host range while others are functional in a limited subset of cell types (for review, see Voss et al., 1986; and Maniatis et al., 1987. For example, the SV40 early gene enhancer is very active in a wide variety of cell types from many mammalian species and has been widely used for the expression of proteins in mammalian cells (Dijkema et al., 1985). Two other examples of promoter/enhancer elements active in a broad range of mammalian cell types are those from the human elongation factor 1 gene (Uetsuki et al., 1989; Kim, et al., 1990; and Mizushima and Nagata, 1990) and the long terminal repeats of the Rous sarcoma virus (Gorman et al., 1982); and the human cytomegalovirus (Boshart et al., 1985).

The term “promoter/enhancer” denotes a segment of DNA capable of providing both promoter and enhancer functions, i.e., the functions provided by a promoter element and an enhancer element as described above. For example, the long terminal repeats of retroviruses contain both promoter and enhancer functions. The enhancer/promoter may be “endogenous” or “exogenous” or “heterologous.” An “endogenous” enhancer/promoter is one that is naturally linked with a given gene in the genome. An “exogenous” or “heterologous” enhancer/promoter is one that is placed in juxtaposition to a gene by means of genetic manipulation (i.e., molecular biological techniques) such that transcription of the gene is directed by the linked enhancer/promoter.

The term “transcription factor binding site” denotes a segment of DNA capable of binding a transcription factor. Such sites are often located within promoter and enhancer elements, but may also be found in other regions of DNA molecules. The interaction of transcription factors with transcription factor binding sites can influence the transcriptional characteristics of a gene. The term “transcription factor binding sequence” denotes a sequence or sequences associated with the binding of transcription factors.

The presence of “splicing signals” on an expression vector often results in higher levels of expression of the recombinant transcript in eukaryotic host cells. Splicing signals mediate the removal of introns from the primary RNA transcript and consist of a splice donor and acceptor site (Sambrook, et al., Molecular Cloning: A Laboratory Manual, 2nd ed., Cold Spring Harbor Laboratory Press, New York, 1989, pp. 16.7-16.8). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are typically a few hundred nucleotides in length. The term “polyadenylation signal”, poly(A) signal” or “poly(A) site” as used herein denotes a genetic element which directs both the termination and polyadenylation of the nascent RNA transcript. The term “poly(A) sequence” as used herein denotes a DNA sequence associated with the termination and polyadenylation of a nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal. The SV40 poly(A) signal is contained on a 237 bp BamH I/Bcl I restriction fragment and directs both termination and polyadenylation (Sambrook, supra, at 16.6-16.7).

Eukaryotic expression vectors may also contain “viral replicons” or “viral origins of replication.” Viral replicons are viral elements which allow for the extrachromosomal replication of a vector in a host cell expressing the appropriate replication factors. Vectors containing either the SV40 or polyoma virus origin of replication replicate to high copy number (up to 104 copies/cell) in cells that express the appropriate viral T antigen. In contrast, vectors containing the replicons from bovine papillomavirus or Epstein-Barr virus replicate extrachromosomally at low copy number (about 100 copies/cell).

The term “in vitro” refers to an artificial environment and to processes or reactions that occur within an artificial environment. In vitro environments include, but are not limited to, test tubes and cell lysates. The term “in vivo” refers to the natural environment (e.g., an animal or a cell) and to processes or reactions that occur within a natural environment. The term “in silico” refers to a computer environment.

The term “sequence identity” means the proportion of base matches between two nucleic acid sequences or the proportion of amino acid matches between two amino acid sequences. Sequence identity is used to refer to a degree of relatedness between two nucleic acid or protein sequences. There may be partial identity or complete identity. Sequence identity is often measured using sequence analysis software, e.g., Sequence Analysis Software Package of the Genetics Computer Group (GCG), 575 Science Drive, Madison, Wis., USA. Such software matches relate sequences by assigning degrees of identity to various substitutions, deletions, insertions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

When sequence identity is expressed as a percentage, e.g., 50%, the percentage denotes the proportion of matches over the length of sequence from one sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence identity between the target nucleic acid and the oligonucleotide sequence is generally not less than 17 target base matches out of 20 possible oligonucleotide base pair matches (85%); preferably not less than 9 matches out of 10 possible base pair matches (90%), and more preferably not less than 19 matches out of 20 possible base pair matches (95%).

Two amino acid sequences share identity if there is a partial or complete identity between their sequences. For example, 85% identity means that 85% of the amino acids are identical when the two sequences are aligned for maximum matching. Gaps (in either of the two sequences being matched) are allowed in maximizing matching; gap lengths of 5 or fewer are preferred with 2 or fewer being more preferred. Alternatively and preferably, two protein sequences (or polypeptide sequences derived from them of at least 100 amino acids in length) share identity, as this term is used herein, if they have an alignment score of more than 5 (in standard deviation units) using the program ALIGN with the mutation data matrix and a gap penalty of 6 or greater. See Dayhoff, M. O., in Atlas of Protein Sequence and Structure, 1972, volume 5, National Biomedical Research Foundation, pp. 101-110, and Supplement 2 to this volume, pp. 1-10. The two sequences or parts thereof more preferably share identity if their amino acids are greater than or equal to 85% identical when optimally aligned using the ALIGN program.

The following terms are used to describe the sequence relationships between two or more polynucleotides: “reference sequence,” “comparison window,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence, i.e., a portion of the complete polynucleotide sequence, that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window,” as used herein, refers to a conceptual segment of at least 20 contiguous nucleotides and wherein the portion of the polynucleotide sequence in the comparison window may comprise additions or deletions, i.e., gaps, of 20 percent or less as compared to the reference sequence (which does not comprise additions or deletions) for optimal alignment of the two sequences.

Methods of alignment of sequences for comparison are well known in the art. Thus, the determination of percent identity between any two sequences can be accomplished using a mathematical algorithm. Preferred, non-limiting examples of such mathematical algorithms are the algorithm of Myers and Miller (1988); the local homology algorithm of Smith and Waterman (1981); the homology alignment algorithm of Needleman and Wunsch (1970); the search-for-similarity-method of Pearson and Lipman (1988); the algorithm of Karlin and Altschul (1990), modified as in Karlin and Altschul (1993).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG)). Alignments using these programs can be performed using the default parameters. The CLUSTAL program is well described by Higgins et al. (1988); Higgins et al. (1989); Corpet et al. (1988); Huang et al. (1992); and Pearson et al. (1994). The ALIGN program is based on the algorithm of Myers and Miller, (1988). The BLAST programs of Altschul et al. (1990), are based on the algorithm of Karlin and Altschul (1993). To obtain gapped alignments for comparison purposes, Gapped BLAST (in BLAST 2.0) can be utilized as described in Altschul et al. (1997). Alternatively, PSI-BLAST (in BLAST 2.0) can be used to perform an iterated search that detects distant relationships between molecules. See Altschul et al., (1990). When utilizing BLAST, Gapped BLAST, PSI-BLAST, the default parameters of the respective programs (e.g. BLASTN for nucleotide sequences, BLASTX for proteins) can be used. See http://www.ncbi.nlm.nih.gov. Alignment may also be performed manually by inspection.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) for the stated proportion of nucleotides over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 60%, preferably at least 65%, more preferably at least 70%, up to about 85%, and even more preferably at least 90 to 95%, more usually at least 99%, sequence identity as compared to a reference sequence over a comparison window of at least 20 nucleotide positions, frequently over a window of at least 20-50 nucleotides, and preferably at least 300 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence.

As applied to polypeptides, the term “substantial identity” means that two peptide sequences, when optimally aligned, such as by the programs GAP or BESTFIT using default gap weights, share at least about 85% sequence identity, preferably at least about 90% sequence identity, more preferably at least about 95% sequence identity, and most preferably at least about 99% sequence identity.

A “partially complementary” sequence is one that at least partially inhibits a completely complementary sequence from hybridizing to a target nucleic acid is referred to using the functional term “substantially identical.” The inhibition of hybridization of the completely complementary sequence to the target sequence may be examined using a hybridization assay (Southern or Northern blot, solution hybridization, and the like) under conditions of low stringency. A substantially identical sequence or probe will compete for and inhibit the binding, i.e., the hybridization, of a completely identical sequence to a target under conditions of low stringency. This is not to say that conditions of low stringency are such that non-specific binding is permitted; low stringency conditions require that the binding of two sequences to one another be a specific, i.e., selective, interaction. The absence of non-specific binding may be tested by the use of a second target that lacks even a partial degree of complementarity, e.g., less than about 30% identity. In this case, in the absence of non-specific binding, the probe will not hybridize to the second non-complementary target.

When used in reference to a double-stranded nucleic acid sequence such as a cDNA or a genomic clone, the term “substantially identical” refers to any probe which can hybridize to either or both strands of the double-stranded nucleic acid sequence under conditions of low stringency as described herein.

“Probe” refers to an oligonucleotide designed to be sufficiently complementary to a sequence in a denatured nucleic acid to be probed (in relation to its length) to be bound under selected stringency conditions.

“Hybridization” and “binding” in the context of probes and denature melted nucleic acid are used interchangeably. Probes which are hybridized or bound to denatured nucleic acid are base paired to complementary sequences in the polynucleotide. Whether or not a particular probe remains base paired with the polynucleotide depends on the degree of complementarity, the length of the probe, and the stringency of the binding conditions. The higher the stringency, the higher must be the degree of complementarity and/or the longer the probe.

The term “hybridization” is used in reference to the pairing of complementary nucleic acid strands. Hybridization and the strength of hybridization, i.e., the strength of the association between nucleic acid strands, is impacted by many factors well known in the art including the degree of complementarity between the nucleic acids, stringency of the conditions involved affected by such conditions as the concentration of salts, the Tm (melting temperature) of the formed hybrid, the presence of other components, e.g., the presence or absence of polyethylene glycol, the molarity of the hybridizing strands and the G:C content of the nucleic acid strands.

The term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “medium” or “low” stringency are often required when it is desired that nucleic acids which are not completely complementary to one another be hybridized or annealed together. The art knows well that numerous equivalent conditions can be employed to comprise medium or low stringency conditions. The choice of hybridization conditions is generally evident to one skilled in the art and is usually guided by the purpose of the hybridization, the type of hybridization (DNA-DNA or DNA-RNA), and the level of desired relatedness between the sequences (e.g., Sambrook et al., 1989; Nucleic Acid Hybridization, A Practical Approach, IRL Press, Washington D.C., 1985, for a general discussion of the methods).

The stability of nucleic acid duplexes is known to decrease with an increased number of mismatched bases, and further to be decreased to a greater or lesser degree depending on the relative positions of mismatches in the hybrid duplexes. Thus, the stringency of hybridization can be used to maximize or minimize stability of such duplexes. Hybridization stringency can be altered by adjusting the temperature of hybridization; adjusting the percentage of helix destabilizing agents, such as formamide, in the hybridization mix; and adjusting the temperature and/or salt concentration of the wash solutions. For filter hybridizations, the final stringency of hybridizations often is determined by the salt concentration and/or temperature used for the post-hybridization washes.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharmacia), 5 g BSA (Fraction V; Sigma)] and 100 g/ml denatured salmon sperm DNA followed by washing in a solution comprising 5×SSPE, 0.1% SDS at 42° C. when a probe of about 500 nucleotides in length is employed.

The term “Tₘ” is used in reference to the “melting temperature.” The melting temperature is the temperature at which 50% of a population of double-stranded nucleic acid molecules becomes dissociated into single strands. The equation for calculating the Tₘ of nucleic acids is well-known in the art. The Tₘ of a hybrid nucleic acid is often estimated using a formula adopted from hybridization assays in 1 M salt, and commonly used for calculating Tₘ for PCR primers: [(number of A+T)×2° C.+(number of G+C)×4° C.]. (C. R. Newton et al., PCR, 2nd Ed., Springer-Verlag (New York, 1997), p. 24). This formula was found to be inaccurate for primers longer than 20 nucleotides. (Id.) Another simple estimate of the Tₘ value may be calculated by the equation: Tₘ=81.5+0.41(% G+C), when a nucleic acid is in aqueous solution at 1 M NaCl. (e.g., Anderson and Young, Quantitative Filter Hybridization, in Nucleic Acid Hybridization, 1985). Other more sophisticated computations exist in the art that take structural as well as sequence characteristics into account for the calculation of Tₘ. A calculated Tₘ is merely an estimate; the optimum temperature is commonly determined empirically.

In the present invention, there may be employed conventional molecular biology and microbiology within the skill of the art. Such techniques are explained fully in the literature. See, e.g., Sambrook, Fritsch & Maniatis, Molecular Cloning: A Laboratory Manual, Third Edition (2001) Cold Spring Harbor Laboratory Press, Cold Spring Harbor, N.Y.

In accordance with the invention, novel nucleic acids have been described. The parent nucleic acid sequence encoding a fluorescent protein has been modified to create synthetic novel forms of the nucleic acid sequences encoding essentially the same fluorescent protein but with enhanced transcriptional and expression properties in the novel host cells, in this case human cells.

1. The Synthetic Nucleic Acid Molecules and Methods of the Invention

The invention provides compositions comprising synthetic nucleic acid molecules that encode fluorescent proteins, as well as methods for preparing those molecules which yield synthetic nucleic acid molecules that are efficiently expressed as a polypeptide or protein with desirable characteristics including reduced inappropriate or unintended transcription characteristics when expressed in a particular cell type.

Natural selection is the hypothesis that genotype-environment interactions occurring at the phenotypic level lead to differential reproductive success of individuals and hence to modification of the gene pool of a population. It is generally accepted that the amino acid sequence of a protein found in nature has undergone optimization by natural selection. However, amino acids exist within the sequence of a protein that do not contribute significantly to the activity of the protein and these amino acids can be changed to other amino acids with little or no consequence. Furthermore, a protein may be useful outside its natural environment or for purposes that differ from the conditions of its natural selection. In these circumstances, the amino acid sequence can be synthetically altered to better adapt the protein for its utility in various applications.

Likewise, the nucleic acid sequence that encodes a protein is also optimized by natural selection. The relationship between coding DNA and its transcribed RNA is such that any change to the DNA affects the resulting RNA. Thus, natural selection works on both molecules simultaneously. However, this relationship does not exist between nucleic acids and proteins. Because multiple codons encode the same amino acid, many different nucleotide sequences can encode an identical protein. A specific protein composed of 500 amino acids can theoretically be encoded by more than 10¹⁵⁰ different nucleic acid sequences.

Natural selection acts on nucleic acids to achieve proper encoding of the corresponding protein. Presumably, other properties of nucleic acid molecules are also acted upon by natural selection. These properties include codon usage frequency, RNA secondary structure, the efficiency of intron splicing, and interactions with transcription factors or other nucleic acid binding proteins. These other properties may alter the efficiency of protein translation and the resulting phenotype. Because of the redundant nature of the genetic code, these other attributes can be optimized by natural selection without altering the corresponding amino acid sequence.

Under some conditions, it is useful to synthetically alter the natural nucleotide sequence encoding a protein to better adapt the protein for alternative applications. A common example is to alter the codon usage frequency of a gene when it is expressed in a foreign host. Although redundancy in the genetic code allows amino acids to be encoded by multiple codons, different organisms favor some codons over others. The codon usage frequencies tend to differ most for organisms with widely separated evolutionary histories. It has been found that when transferring genes between evolutionarily distant organisms, the efficiency of protein translation can be substantially increased by adjusting the codon usage frequency (see U.S. Pat. Nos. 5,096,825, 5,670,356 and 5,874,304).

Because of the evolutionary distance, the codon usage of genes that encode fluorescent proteins may not correspond to the optimal codon usage of the experimental cells. Examples include green fluorescent protein (GFP) reporter genes, which are derived from coelenterates but are commonly used in plant and mammalian cells. To achieve sensitive quantitation of fluorescent protein gene expression, the activity of the gene product must not be endogenous to the experimental host cells. Thus, fluorescent protein genes are usually selected from organisms having unique and distinctive phenotypes. Consequently, these organisms often have widely separated evolutionary histories from the experimental host cells.

Previously, to create genes having a more optimal codon usage frequency but still encoding the same gene product, a synthetic nucleic acid sequence was made by replacing existing codons with codons that were generally more favorable to the experimental host cell (see, e.g., U.S. Pat. Nos. 5,096,825, 5,670,356 and 5,874,304.) The result was a net improvement in codon usage frequency of the synthetic gene. However, the optimization of other attributes was not considered and so these synthetic genes likely did not reflect genes optimized by natural selection.

In particular, improvements in codon usage frequency are intended only for optimization of an RNA sequence based on its role in translation into a protein. Thus, previously described methods did not address how the sequence of a synthetic gene affects the role of DNA in transcription into RNA. Most notably, consideration had not been given as to how transcription factors may interact with the synthetic DNA and consequently modulate or otherwise influence gene transcription. For genes found in nature, the DNA would be optimally transcribed by the native host cell and would yield an RNA that encodes a properly folded gene product. In contrast, synthetic genes have previously not been optimized for transcriptional characteristics. Rather, this property has been ignored or left to chance.

This concern is important for all genes, but particularly important for reporter genes, which are most commonly used to quantitate transcriptional behavior in the experimental host cells. Hundreds of transcription factors have been identified in different cell types under different physiological conditions, and likely more exist but have not yet been identified. All of these transcription factors can influence the transcription of an introduced gene. The product of a useful synthetic reporter gene of the invention has a minimal risk of influencing or perturbing intrinsic transcriptional characteristics of the host cell because the structure of that gene has been altered. A particularly useful synthetic reporter gene will have desirable characteristics under a new set and/or a wide variety of experimental conditions. To best achieve these characteristics, the structure of the synthetic gene should have minimal potential for interacting with transcription factors within a broad range of host cells and physiological conditions. Minimizing potential interactions between a reporter gene and a host cell's endogenous transcription factors increases the value of a reporter gene by reducing the risk of inappropriate transcriptional characteristics of the gene within a particular experiment, increasing applicability of the gene in various environments, and increasing the acceptance of the resulting experimental data.

These concerns are also important for fluorescent protein genes, which may be used to quantitate transcriptional behavior and are frequently used as a qualitative measure or as a fusion with another protein to monitor the movement or localization of the fused protein. As described hereinabove, hundreds of transcription factors may be present in a host cell and can influence the transcription of an introduced gene. A useful synthetic fluorescent protein gene of the invention has a minimal risk of influencing or perturbing intrinsic transcriptional characteristics of the host cell because the structure of that gene has been altered. A particularly useful synthetic fluorescent protein gene will have desirable characteristics under a new set and/or a wide variety of experimental conditions. To best achieve these characteristics, the structure of the synthetic fluorescent protein gene should have minimal potential for interacting with transcription factors within a broad range of host cells and physiological conditions. Minimizing potential interactions between a fluorescent protein gene and a host cell's endogenous transcription factors increases the value of a fluorescent protein gene by reducing the risk of inappropriate transcriptional characteristics of the gene within a particular experiment, increasing applicability of the gene in various environments, and increasing the acceptance of the resulting experimental data.

In contrast, a reporter gene comprising a native nucleotide sequence, based on a genomic or cDNA clone from the original host organism, may interact with transcription factors when expressed in an exogenous host. This risk stems from two circumstances. First, the native nucleotide sequence contains sequences that were optimized through natural selection to influence gene transcription within the native host organism. However, these sequences might also influence transcription when the gene is expressed in exogenous hosts, i.e., out of context, thus interfering with its performance as a reporter gene. Second, the nucleotide sequence may inadvertently interact with transcription factors that were not present in the native host organism, and thus did not participate in its natural selection. The probability of such inadvertent interactions increases with greater evolutionary separation between the experimental cells and the native organism of the reporter gene.

Likewise, a fluorescent protein gene comprising a native nucleotide sequence, based on a genomic or cDNA clone from the original host organism or a mutant of the originally isolated fluorescent protein, may interact inappropriately with transcription factors when expressed in an exogenous host, as described hereinabove. The probability of such inadvertent interactions increases with greater evolutionary separation between the experimental cells and the native organism of the reporter gene.

These potential interactions with transcription factors would likely be disrupted when using a synthetic fluorescent protein gene having alterations in codon usage frequency. However, a synthetic fluorescent protein gene sequence, designed by choosing codons based only on codon usage frequency, is likely to contain other unintended transcription factor binding sites since the synthetic gene has not been subjected to the benefit of natural selection to correct inappropriate transcriptional activities. Inadvertent interactions with transcription factors could also occur whenever the encoded amino acid sequence is artificially altered, e.g., to introduce amino acid substitutions. Similarly, these changes have not been subjected to natural selection, and thus may exhibit undesired characteristics.

Thus, the invention provides a method for preparing synthetic nucleic acid sequences that reduces the risk of undesirable interactions of the nucleic acid with transcription factors when expressed in a particular host cell, thereby reducing inappropriate or unintended transcriptional characteristics. Preferably, the method yields synthetic genes containing improved codon usage frequencies for a particular host cell and with a reduced occurrence of vertebrate transcription factor binding sequences. The invention also provides a method of preparing synthetic genes containing improved codon usage frequencies with a reduced occurrence of transcription factor binding sequences and additional beneficial structural attributes. Such additional attributes include the absence of inappropriate RNA splicing sequences, poly(A) addition sequences, undesirable restriction sequences, ribosomal binding sequences, and secondary structural motifs such as hairpin loops.

Thus, the nucleic acid of the invention provides novel synthetic nucleic acid sequences encoding fluorescent proteins that reduce the risk of undesirable interactions of the nucleic acid with transcription factors when expressed in a particular host cell. Preferably, the method yields synthetic fluorescent protein genes containing improved codon usage frequencies for a particular host cell and with a reduced occurrence of transcription factor binding sequences. The invention also provides a method of preparing synthetic fluorescent protein genes containing improved codon usage frequencies with a reduced occurrence of transcription factor binding sequences and additional beneficial structural attributes, as named above. Such additional attributes include, but are not limited to, the absence of inappropriate RNA splicing sequences, poly(A) addition sequences, undesirable restriction sequences, ribosomal binding sequences, and secondary structural motifs such as hairpin loops.

Also provided is a method for preparing synthetic genes encoding the same or highly similar proteins (“codon distinct” versions). Preferably, the synthetic genes have a differing ability to hybridize to a common polynucleotide probe sequence, or have a reduced risk of recombining when present together in living cells. To detect recombination, PCR amplification of the reporter sequences using primers complementary to flanking sequences and sequencing of the amplified sequences may be employed. Thus provided is a method for preparing synthetic genes encoding the same or highly similar fluorescent proteins (“codon distinct” versions). Preferably, the synthetic fluorescent protein genes have a differing ability to hybridize to a common polynucleotide probe sequence, or have a reduced risk of recombining when present together in living cells. To detect recombination, PCR amplification of the reporter sequences using primers complementary to flanking sequences and sequencing of the amplified sequences may be employed.

To select codons for the synthetic nucleic acid molecules of the invention, preferred codons have a relatively high codon usage frequency in a selected host cell, and their introduction results in the introduction of relatively few transcription factor bindingsequences, relatively few other undesirable structural attributes, and optionally a characteristic that distinguishes the synthetic gene from another gene encoding a highly similar protein. Thus, the synthetic nucleic acid product obtained by the method of the invention is a synthetic gene with improved level of expression due to improved codon usage frequency, a reduced risk of inappropriate transcriptional behavior due to a reduced number of undesirable transcription regulatory sequences, and optionally any additional characteristic due to other criteria that may be employed to select the synthetic sequence.

Optimally, at least one characteristic in the synthetic gene is enhanced protein expression in the desired host cell vis-a-vis the native host cell. Thus, the synthetic nucleic acid product obtained by the method of the invention is a synthetic fluorescent protein gene with improved level of expression due to improved codon usage, a reduced risk of inappropriate transcriptional behavior due to a reduced number of undesirable transcription regulatory sequences, and optionally any additional characteristic due to other criteria that may be employed to select the synthetic sequence.

The invention may be employed with any nucleic acid sequence, e.g., a native sequence such as a cDNA or one which has been manipulated in vitro, e.g., to introduce specific alterations such as the introduction or removal of a restriction enzyme recognition sequence, the alteration of a codon to encode a different amino acid or to encode a fusion protein, increased brightness, or to alter GC or AT content (% of composition) of nucleic acid molecules. Moreover, the method of the invention is useful with any gene, but particularly useful for reporter genes as well as other genes associated with the expression of reporter genes, such as selectable markers. Preferred genes include, but are not limited to, those encoding lactamase (β-gal), neomycin resistance (Neo), CAT, GUS, galactopyranoside, xylosidase, thymidine kinase, arabinosidase, fluorescent proteins, and the like.

Moreover, the method of the invention is useful with any fluorescent protein gene. Preferred genes include, but are not limited to, those encoding GFP and red fluorescent protein (RFP), and the like. Elements of the present disclosure are exemplified in detail through the use of particular fluorescent protein genes. Of course, many examples of suitable fluorescent protein genes are known to the art and can be employed in the practice of the invention. Therefore, it will be understood that the following discussion is exemplary rather than exhaustive. In light of the techniques disclosed herein and the general recombinant techniques that are known in the art, the present invention renders possible the alteration of any fluorescent protein gene. Exemplary fluorescent protein genes include, but are not limited to, a GFP originally isolated from Montastraea cavernosa and RFP originally isolated from a polyp believed to be either Actinodiscus or Discosoma.

As used herein, a “marker gene” or “reporter gene” is a gene that imparts a distinct phenotype to cells expressing the gene and thus permits cells having the gene to be distinguished from cells that do not have the gene. Such genes may encode either a selectable or screenable marker, depending on whether the marker confers a trait which one can “select” for by chemical means, i.e., through the use of a selective agent (e.g., a herbicide, antibiotic, or the like), or whether it is simply a “reporter” trait that one can identify through observation or testing, i.e., by “screening.” Elements of the present disclosure are exemplified in detail through the use of particular marker genes. Of course, many examples of suitable marker genes or reporter genes are known to the art and can be employed in the practice of the invention. Therefore, it will be understood that the following discussion is exemplary rather than exhaustive. In light of the techniques disclosed herein and the general recombinant techniques, which are known in the art, the present invention renders possible the alteration of any gene.

The method of the invention can be performed by, although it is not limited to, a recursive process. The process includes assigning preferred codons to each amino acid in a target molecule, e.g., a parent nucleotide sequence, based on codon usage in a particular species, identifying potential transcription regulatory sequences such as transcription factor binding sequences in the nucleic acid sequence having preferred codons, e.g., using a database of such binding sequences, optionally identifying other undesirable sequences, and substituting an alternative codon (i.e., encoding the same amino acid) at positions where undesirable transcription factor binding sequences or other sequences occur. For codon distinct versions, alternative preferred codons are substituted in the attempt to reduce the number or type of transcriptional factor binding sequences for each version. If necessary, the identification and elimination of potential transcription factor or other undesirable sequences can be repeated until a nucleotide sequence is achieved containing a maximum number of preferred codons and a minimum number of undesired sequences including transcription regulatory sequences or other undesirable sequences. Also, optionally, desired sequences, e.g., restriction enzyme recognition sequences, can be introduced. After a synthetic nucleic acid molecule is designed and constructed, its properties relative to the parent nucleic acid sequence can be determined by methods well known to the art. For example, the expression of the synthetic and parent nucleic acid molecules in a series of vectors in a particular cell can be compared.

Thus, generally, the method of the invention comprises identifying a target nucleic acid sequence that encodes a fluorescent protein, and a host cell of interest, for example, a plant (dicot or monocot), fungus, yeast, or mammalian cell. Preferred host cells are mammalian host cells such as CHO, COS, 293, Hela, CV-1 and NIH3T3 cells. Based on preferred codon usage in the host cell(s) and, optionally, low codon usage in the host cell(s), e.g., high usage mammalian codons and low usage E. coli and mammalian codons, codons to be replaced are determined. Codon distinct versions of two synthetic nucleic acid molecules may be determined using alternative preferred codons are introduced to each version. Thus, for amino acids having more than two codons, one preferred codon is introduced to one version and another preferred codon is introduced to the other version. For amino acids having more than one codon, the two codons with the largest number of mismatched bases may be identified and one is introduced to one version and the other codon is introduced to the other version. Concurrent, subsequent, or prior to selecting codons to be replaced, desired and undesired sequences, such as undesired transcriptional regulatory sequences, in the target sequence are identified. These sequences can be identified using databases and software such as EPD, NNPD, REBASE, TRANSFAC, TESS, GenePro, MAR (www.ncgr.org/MAR-search) and BCM Gene Finder, further described herein. After the sequences are identified, the modification(s) are introduced. Once a desired synthetic nucleic acid sequence is obtained, it can be prepared by methods well known to the art (such as PCR with overlapping primers or commercial gene synthesis), and its structural and functional properties compared to the target nucleic acid sequence, including, but not limited to, percent identity, presence or absence of certain sequences, for example, restriction sequences, percent of codons changed (such as an increased or decreased usage of certain codons) and expression rates.

In a certain preferred embodiment, the following steps are performed.

1. The codon usage of a parent gene, or portion of a gene, is optimized for expression in one or more foreign hosts preferably without altering the amino acid sequence.

2. Optionally, desired nucleotide sequences (e.g., Kozak consensus sequences, specific binding sequences, restriction enzyme sequences, and recombination sequences) are introduced by altering the gene sequence and, if required, also the amino acid sequence.

3. Undesired transcription regulatory sequences and restriction enzyme recognition sequences are identified by locating descriptions of such sequences within the gene sequence.

Such descriptions may be specific individual sequence descriptions, consensus sequence descriptions, matrix descriptions, or others. The descriptions may be obtained from own research, literature, or other public or commercial sources. The descriptions can be located in the gene sequence using different search methods, for example, search by eye, text searches, sequence analysis software, or specialized software such as MatInspector professional. The person skilled in the art will understand how to select parameters applicable to the method used that will yield the desired results.

4. Undesired transcription regulatory sequences and restriction enzyme recognition sequences are then eliminated from the gene sequence by replacing one or more codons with alternate codons for the same amino acid. To remove highly undesired sequences, the user might choose to substitute codons that that are not favored in the selected foreign host, or that alter the amino acid sequence if this does not unduly compromise the desired properties of the polypeptide. Replacement codons or codon combinations that introduce new undesired transcription regulatory sequences or restriction enzyme recognition sequences should be avoided. Out of the possible replacement codons or codon combinations, those that most completely remove undesired transcription regulatory sequences are preferred. Replacement of many codons that are non-preferred for the selected foreign host(s) should be avoided. Codon replacements can be selected and introduced manually or with the help of software such as SequenceShaper. The person skilled in the art will understand how to select parameters applicable to the method used that will yield the desired results.

5. Steps 3 and 4 may be repeated if desired or needed with adjusted parameters until a final sequence is obtained that contains as few undesired transcription regulatory sequences and restriction enzyme recognition sequences as possible or acceptable.

6. The final designed nucleic acid sequence may then be synthesized/constructed and cloned in a suitable genetic vector. The genetic vector may be an expression vector to allow protein transcription of the synthesized gene in the selected foreign host(s) or other appropriate host.

As described below, the method was used to create a synthetic gene encoding a green fluorescent protein (GFP) that was a mutated form of a GFP originally isolated from Montastraea cavernosa. The synthetic gene supports much greater levels of fluorescence in a host cell when compared to the parent GFP. In addition, it is expected that there will be decreased anomalous expression of the synthetic GFP when compared to the parent GFP.

Exemplary Uses of the Molecules of the Invention

The synthetic genes of the invention preferably encode the same proteins as their parental counterpart (or nearly so), and, when compared to the parent protein, have improved codon usage while being largely devoid of known transcription regulatory sequences in the coding region. (It is recognized that a small number of amino acid changes may be desired to enhance a property of the native counterpart protein, e.g. to enhance the fluorescent properties of a fluorescent protein.) This increases the level of expression of the protein encoded by the synthetic gene and reduces the risk of anomalous expression of the protein. For example, studies of many important events of gene regulation, which may be mediated by weak promoters, are limited by insufficient reporter signals from inadequate expression of the reporter proteins. The synthetic fluorescent protein genes described herein permit detection of weak promoter activity because of the large increase in level of expression, which enables increased detection sensitivity. A further benefit is that transcription factors that may be available in limited quantities are not utilized by the cell in non-productive binding events. Also, the use of some selectable markers may be limited by the expression of that marker in an exogenous cell. Thus, synthetic selectable marker genes which have improved codon usage for that cell, and have a decrease in other undesirable sequences, (e.g., transcription factor binding sequences), can permit the use of those markers in cells that otherwise were undesirable as hosts for those markers.

Promoter crosstalk is another concern when a co-reporter gene is used to normalize transfection efficiencies. With the enhanced expression of synthetic genes, the amount of DNA containing strong promoters can be reduced, or DNA containing weaker promoters can be employed, to drive the expression of the co-reporter. In addition, there may be a reduction in the background expression from the synthetic reporter genes of the invention. This characteristic makes synthetic reporter genes more desirable by minimizing the sporadic expression from the genes and reducing the interference resulting from other regulatory pathways.

The use of reporter genes in imaging systems, which can be used for in vivo biological studies or drug screening, is another use for the synthetic genes of the invention. Due to their increased level of expression, the protein encoded by a synthetic gene is more readily detectable by an imaging system. In the case of a fluorescent protein encoded by a synthetic gene, during fluorescence activated cell sorting (FACS), fluorescence intensity may be increased or reduced, according to need of the investigator. In addition, the synthetic fluorescent protein genes may be used to express fusion proteins, for example fusions with secretion leader sequences or cellular localization sequences, to study transcription in difficult-to-transfect cells such as primary cells, and/or to improve the analysis of regulatory pathways and genetic elements. Further, synthetic fluorescent protein genes may be fused to a gene of interest such that expression of the gene of interest can be tracked, e.g., inside a host cell.

Other uses include, but are not limited to, the detection of rare events that require extreme sensitivity (e.g., studying RNA recoding), use with internal ribosome entry sites (IRES), to improve the efficiency of in vitro translation or in vitro transcription-translation coupled systems such as TNT™ (Promega Corp., Madison, Wis.), study of fluorescent proteins optimized to different host organisms (e.g., plants, fungi, and the like). In addition, the synthetic fluorescent proteins of the invention can be used as reporters. Thus, the fluorescent proteins can be used as reporter molecules in multiwell assays, and as reporter molecules in drug screening with the advantage of minimizing possible interference of reporter signal by different signal transduction pathways and other regulatory mechanisms. Multiple synthetic fluorescent protein genes can be used as co-reporters to, e.g., monitor drug toxicity.

Additionally, uses for the nucleic acid molecules of the invention include, but are not limited to, fluorescent microscopy, to detect and/or measure the level of gene expression in vitro and in vivo, (e.g., to determine promoter strength), subcellular localization or targeting (fusion protein), as a marker, in calibration, in a kit, (e.g., for dual assays), for in vivo imaging, to analyze regulatory pathways and genetic elements, and in multi-well formats.

Demonstration of the Invention Using a Green Fluorescent Protein Gene

The gene for Green II, a mutant green fluorescent protein generated from a wild type gene isolated from Montastraea cavernosa, was used to demonstrate the invention. Green II has a high resistance to photobleaching. Therefore, it can be useful in, e.g., cell monitoring. Photobleaching is a light induced change in a fluorophore, resulting in the loss of absorption of light of a particular wavelength by the fluorophore and the loss of fluorescence of the fluorophore. This property can limit the usefulness of some fluorescent proteins, e.g. by reducing time available to photograph or to observe specimens. Hence, a fluorescent protein that has a high resistance to photobleaching can be beneficial in situations where prolonged fluorescence is desired.

The following Examples are provided for illustrative purposes only. The Examples are included herein solely to aid in a more complete understanding of the presently described invention. The Examples do not limit the scope of the invention described or claimed herein in any fashion.

### EXAMPLE 1

### Synthetic Green Fluorescent Protein Nucleic Acid Molecules

McGFP is a green fluorescent protein (GFP) that was isolated from Montastraea cavernosa. McGFP was mutated during a first round of low stringency PCR to induce mutations in the wild type gene. From the first round of PCR, Green I was produced. Green I had higher relative fluorescence intensity than the wild type GFP. Green I was mutated during a second round of low stringency PCR performed on the DNA encoding Green I to generate Green II. When compared to the DNA sequence encoding the Green I, the DNA encoding Green II contains a single nucleotide change: a cytosine to thymine mutation at nucleotide 527. This results in an S at position 176 in Green I, and an F at the same position in Green II. Green II had a high resistance to photobleaching.

Green II was used as a parent gene in humanization of the nucleic acid sequences. A synthetic gene sequence was designed in silico using the following software tools: MatInspector professional Release 5.2 with Matrix Family Library Ver 2.3 and 2.4, ModelInspector professional Release 4.7.8 and 4.7.9 with Promoter Module Library Ver 2.2 and 2.3, and SequenceShaper Release 2.3 (all from Genomatix Software GmbH, Munich, Germany). The gene was designed to 1) have optimized codon usage for expression in mammalian cells, 2) have a reduced number of transcriptional regulatory sequences including vertebrate transcription factor binding sequences, splice sequences, poly(A) addition sequences and promoter sequences, as well as prokaryotic (e.g., E. coli) regulatory sequences, 3) have a Kozak sequence, 4) have at least one novel restriction enzyme recognition sequence for cloning, and 5) be devoid of unwanted restriction enzyme recognition sequences, e.g., those which are likely to interfere with standard cloning procedures.

Not all design criteria could be met equally well at the same time. The following priority was established: elimination of vertebrate transcription factor (TF) binding sequences received the highest priority, followed by elimination of splice sequences and poly(A) addition sequences, and finally elimination of prokaryotic regulatory sequences. When removing regulatory sequences, the strategy was to work from the lesser important to the most important to ensure that the most important changes were made last, and inadvertent changes to these improvements did not occur. Then the sequence was rechecked for the appearance of new lower priority sequences and additional changes made as needed. Thus, the process for designing a synthetic gene sequence, using computer programs described herein, involves optionally iterative steps that are detailed below.

MatInspector professional employs matrix descriptions of transcription factor binding sequences to locate these sequences within a DNA sequence. The matrix descriptions are contained within a transcription factor weight matrix database (a library of matrix descriptions for transcription factor binding sequences). Methods for MatInspector were originally described in Quandt et al. 1995 (Quandt, K., Frech, K., Karas, H., Wingender, E., Werner, T. (1995). MatInd and MatInspector: new fast and versatile tools for detection of consensus matches in nucleotide sequence data. Nucleic Acids Res. 1995, vol. 23, 4878-4884.).

Within the transcription factor weight matrix database, the matrix descriptions are divided into categories (e.g., transcription factor binding sequences from fungi, insects, plants, vertebrates, etc.). Each matrix description belongs to a matrix family, where similar and/or related matrix descriptions are grouped together, to eliminate redundant matches by MatInspector Professional. Users can add their own matrix descriptions for transcription factor binding sequences or other sequences, such as other transcription regulatory sequences or restriction enzyme sequences. The database versions used in this Example were Matrix Family Library Ver 2.3 (which contains 264 vertebrate matrix descriptions in 103 families) and Ver 2.4 (which contains 275 vertebrate matrix descriptions in 106 families).

To perform a search with MatInspector professional, the user may define and save a subset of matrix descriptions to be used for the search. In addition, the user may define the threshold scoring parameters “core similarity” and “matrix similarity” for each matrix description used in a search. The “core sequence” is defined as the highest conserved positions, typically four, within the matrix description. The core and matrix similarity scores are calculated as described in Quandt et al. 1995. A perfect match to the matrix description gets a score of 1.00 (each sequence position corresponds to the highest conserved nucleotide at that position in the matrix description); a “good” match to the matrix description usually has a similarity score of >0.80. Mismatches in highly conserved positions of the matrix description decrease the matrix similarity score more than mismatches in less conserved regions. An “Optimized” matrix similarity scoring threshold, designed to minimize false positives and false negatives, is supplied for each individual matrix description in the transcription factor weight matrix database (and is automatically calculated for user-defined matrices).

The user-defined matrix subset and its matrix scoring parameters (denoted as “core similarity threshold/matrix similarity threshold”) used for analysis of sequences described in this Example are shown below. Changes to this subset are noted in the individual design steps. This subset contains all vertebrate matrix families (ALL vertebrates.lib), and a number of user-defined matrix families (U$), whose IUPAC (International Union of Pure and Applied Chemistry) consensus sequences are shown below where appropriate. The matrix descriptions of eukaryotic splice donor (5′, “Splice-A”) and acceptor (3′, “Splice-D”) sequences were generated based on Lodish et al. 2000 (Molecular Cell Biology, 4^{th} Edition, Lodish et. al. 2000, p.416) and Alberts et al. 1994 (Molecular Biology of the Cell, 3^{rd} Edition, 1994, Alberts et al., p.373). The matrix description for the Kozak sequence was generated based on Kozak 1987 (An analysis of 5′-noncoding sequences from 699 vertebrate messenger RNAs. Nucleic Acids Research, 1987, Vol. 15, p. 8125). The matrix descriptions of two poly(A) sequences were based on Tabaska 1999 (Detection of polyadenylation signals in human DNA sequences, Tabaska J E, Zhang M Q. Gene 1999, 231 (1-2):77-86). The matrix descriptions of E. coli ribosome binding sequences (“EC-RBS”) were generated based on Glass RE 1992 (Gene Functions: E. coli and its heritable elements. University of California Press, 1982, Robert E. Glass, p.95) and Ringquist 1992 (Translation Initiation in Escherichia coli; Sequences Within the Ribosome Binding Site. Ringquist, Steven, et al., Molecular Microbiology, 1992, 6(9), p.1221). The matrix descriptions of E. coli promoter-10 and -35 sequences (“EC-P-10” and “EC-P-35”) and complete E. coli promoter sequences, i.e. -35 and -10 sequences separated by spacer sequences of 16, 17, or 18 nucleotides (“EC-Prom”), were generated based on Lisser et al. 1993 (Compilation of E. coli mRNA promoter sequences. S. Lisser and H. Margalit, Nucleic Acids Research 1993, Vol 21, Issue 7, p.1512). Restriction enzyme recognition sequences can be easily found in the catalogs of biological reagent supply companies such as Promega Corporation or in databases such as Rebase™ (http://rebase.neb.com/rebase/rebase.html).

The matrix scoring parameters for each matrix description in the user-defined matrix subsets were chosen to match the design criteria for the sequence of interest. We chose scoring parameters (0.75/Optimized) for identifying vertebrate transcription factor binding sequences and more stringent scoring parameters (i.e., increased core and/or matrix similarity) for some user-defined transcription regulatory sequences. Restriction enzyme recognition sequences were assigned a matrix similarity threshold of 1.00 since only perfect matches to the matrix are of interest.

User-defined matrix subset

ALL vertebrates.lib
(0.75/Optimized)

| | |
|---|---|
| U$Splice-A (1.00/Optimized) | IUPAC “ynCAGR” |
| U$Splice-D (1.00/ Optimized) | IUPAC “mAGGTragt” |
| U$PolyAsig (1.00/1.00) | IUPAC “AATAAA”, “ATTAAA” |
| U$Kozak (0.75/Optimized) | IUPAC “nnnnnnnncrmCATGn” |
| U$EC-RBS (1.00/1.00) | IUPAC “AAGG”, “AGGA”, “GGAG”, |
| | “GAGG” |
| U$EC-P-10 (1.00/Optimized) | IUPAC “TATAat” |
| U$EC-P-35 (1.00/Optimized) | IUPAC “TTGAca” |
| U$EC-Prom (1 .00/Optimized) | IUPAC “ttgacn(n)_{15,16,17} TATAat” |
| U$AccI (0.75/1.00) | |
| U$BamHI (0.75/1.00) | |
| U$BglII (0.75/1.00) | |
| U$ClaI (0.75/1.00) | |
| U$EcoRI (0.75/1.00) | |
| U$EcoRV (0.75/1.00) | |
| U$MluI (0.75/1.00) | |
| U$NaeI (0.75/1.00) | |
| U$NcoI (0.75/1.00) | |
| U$NheI (0.75/1.00) | |
| U$NotI (0.75/1.00) | |
| U$SalI (0.75/1.00) | |
| U$SmaI (0.75/1.00) | |
| U$XbaI (0.75/1.00) | |
| U$XhoI (0.75/1.00) | |

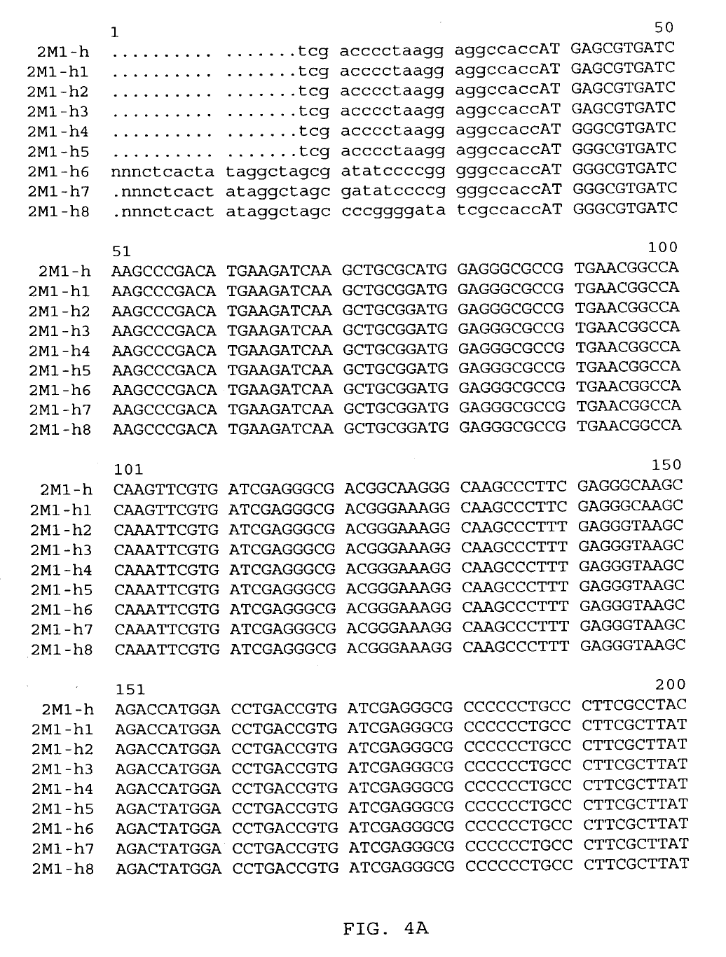

When using a program such as MatInspector professional for identification of transcription regulatory sequences or restriction enzyme recognition sequences in a sequence of interest, it is preferable to also include, 5′ and 3′ flanking DNA sequences in addition to the actual sequence of interest. Examples of flanking DNA sequences include sequences that would be expected if the sequence of interest were cloned into an expression vector, and/or a short ambiguous DNA sequence, for example “NNN”. This makes it less likely that the search algorithm will fail to detect, e.g., transcription regulatory sequences that overlap or are flush with the 5′ or 3′ end of the sequence of interest. In this Example, the gene sequence (ORF) contained 5′ and 3′ flanking DNA sequences. Flanking sequences used in this Example are shown in FIGS. 4A-4D as small case letters.

After identification of transcription regulatory sequences or restriction enzyme recognition sequences in a sequence with MatInspector professional, one or more of these sequences are eliminated by substituting alternate codons encoding the same amino acid either manually or with help of a software tool. It must be appreciated that the elimination of one transcription regulatory sequence or restriction enzyme recognition sequence may cause inadvertent introduction of yet one or more new ones. Thus, the process of identifying and eliminating transcription regulatory sequences or restriction enzyme recognition sequence is often iterative to achieve an optimal sequence.

In this Example we used SequenceShaper, a software tool that allows elimination of transcription factor binding sequences or other user-defined sequences. It allows the simultaneous deletion of several sequences identified with MatInspector professional without introducing new sequences (based on the user-defined matrix subset used in the MatInspector step) or making changes to the encoded polypeptide. For each sequence selected for elimination, a list of possible mutations restricted by user-defined parameters is created. The standard parameters we used, unless noted otherwise, were:

SequenceShaper standard parameters:

Remaining threshold: 0.70 core similarity/Optimized-0.20 matrix similarity (default)

Don't insert additional site

Conserve open reading frame (ORF)

The “remaining threshold” specifies the score each identified sequence may have after the mutations were introduced. If no possible mutations are found, these thresholds should be increased. “Don't insert additional site” prevents generation of additional sequences contained in the user-defined subset used for identification of sequences with MatInspector professional. “Conserve open reading frame (ORF)” allows only mutations to be suggested which do not influence the amino acids coded by the sequence. From the list of possible mutations we preferably selected those that will introduce preferred codons. E. coli ribosome binding sequences in the minus strand and those not followed by a methionine codon less than 21 bases downstream were ignored. Some transcription regulatory sequences or restriction enzyme recognition sequences might be impossible to remove without introducing a new transcription regulatory sequences or restriction enzyme recognition sequences. In such a case a decision was made to keep whichever sequence best matched the stated design criteria.

Additional analyses were performed using ModelInspector professional. This software tool employs a library of experimentally verified promoter modules to locate regions in a DNA sequence that contain two or more transcription factor binding sequences having a defined relative distance and orientation. (Frech, K. et. al, A novel method to develop highly specific models for regulatory units detects a new LTR in GenBank which contains a functional promoter. J. Mol. Biol., 1997, 270 (5), 674-687).

The process for designing the synthetic hGreen II gene sequence, using the computer programs described herein, involved several optionally iterative steps that are detailed below.

1. The codon usage of the parent gene (Green II; (SEQ. ID. NO:21))) coding region was optimized for expression in mammalian cells without altering the amino acid sequence, and flanking sequences were added to the 5′ and 3′ ends of the coding region (creating 2M 1-h (SEQ. ID. NO:3)).

2. Sequence 2M1-h was analyzed for transcription regulatory sequences and restriction enzyme sequences using MatInspector professional with Matrix Family Library Ver 2.3 and User-defined matrix subset (without NcoI, NaeI, Kozak, PolyAsig).

3. As many undesirable sequences as possible were removed, following the above criteria with SequenceShaper standard parameters (creating sequence 2M1-h1 (SEQ. ID. NO:5)).

4. Additional undesirable sequences were removed with SequenceShaper, increasing the matrix similarity threshold to Optimized-0.01 (creating 2M1-h2 (SEQ. ID. NO:7)).

5. Additional undesirable sequences were removed with SequenceShaper, increasing the core similarity threshold to 0.75 and the matrix similarity threshold to Optimized-0.01 (creating 2M1-h3 (SEQ. ID. NO:9)).

6. Sequence 2M1-h3 was also analyzed for the presence of promoter modules and genomic repeats using ModelInspector professional Release 4.7.8 with Promoter Module Library Ver 2.2 and Genomic Repeat Library Ver 1.0 using default parameters. No promoter modules or genomic repeats were found.

7. Sequence 2M1-h3 was modified by changing the serine codon (AGC) at amino acid position 2 to a glycine codon (GGC) to better match the Kozak consensus sequence; this also introduced an NcoI restriction enzyme sequence overlapping with the 5′ end of the gene sequence (creating sequence 2M1-h4 (SEQ. ID. NO:11)).

8. Sequence 2M1-h4 was analyzed for transcription regulatory sequences and restriction enzyme sequences using Matlnspector professional with Matrix Family Library Ver 2.3 and User-defined matrix subset (without Nael, Kozak, PolyAsig).

9. An internal NcoI sequence was removed from sequence 2M1-h4 with SequenceShaper standard parameters (creating sequence 2M1-h5 (SEQ. ID. NO:13)).

10. Sequence 2M1-h5 was analyzed for the presence of promoter modules and genomic repeats using ModelInspector professional Release 4.7.8 with Promoter Module Library Ver 2.2 and Genomic Repeat Library Ver 1.0 using default parameters. No promoter modules or genomic repeats were found.

11. Sequence 2M1-h5 was further modified by changing the 5′ and 3′ flanking regions, and by changing the lysine codon at position 227 (AAG) to a glycine codon (GGC) to introduce a new NaeI restriction enzyme sequence, providing a cloning sequence for the creation of, e.g., fusion proteins (creating sequence 2M1-h6 (SEQ. ID. NO: 15)).

12. Sequence 2M1-h6 was analyzed for transcription regulatory sequences and restriction enzyme sequences using MatInspector professional with Matrix Family Library Ver 2.4 and User-defined matrix subset (without NaeI). Several new transcription factor binding sequences were identified due to the updated Matrix Family Library.

13. Sequence 2M1-h6 was analyzed for the presence of promoter modules and genomic repeats using ModelInspector professional Release 4.7.9 with Promoter Module Library Ver 2.3 and Genomic Repeat Library Ver 1.0 using default parameters. No promoter modules or genomic repeats were found.

14. Sequence 2M1-h6 was further modified by changing the 5′ flanking region (creating sequence 2M1-h7 (SEQ. ID. NO:17)).

15. Sequence 2M1-h7 was analyzed for transcription regulatory sequences and restriction enzyme sequences using MatInspector professional with Matrix Family Library Ver 2.4 and User-defined matrix subset.

16. As many sequences as possible were removed with SequenceShaper, first using standard parameters and then using less stringent parameters (Remaining threshold: 0.75 core similarity/Optimized-0.01 matrix similarity).

17. To remove remaining undesired transcriptional regulatory sequences from 2M1-h7, the previous two steps were repeated using a User-defined matrix subset containing only the vertebrate transcription factor binding sequences and restriction enzyme recognition sequences. This allowed removal of additional high priority transcriptional regulatory sequences by introducing additional lower priority sequences, e.g. E. coli ribosome binding and promoter sequences, splice donor and acceptor sequences, and poly(A) sequences (creating sequence 2M1-h8 (SEQ. ID. NO:19); the gene coding region of which is called hGreen II).

18. Sequence 2M1-h8 was analyzed for the presence of promoter modules and genomic repeats using ModelInspector professional Release 4.7.9 with Promoter Module Library Ver 2.3 using default parameters. No promoter modules were found.

19. The sequence of 2M1-h8, excluding the 5′ and 3′ NNNs, was synthesized by Blue Heron Biotechnology, Inc. (22310 20^{th} Avenue SE #100, Bothell, Wash. 98021) using its proprietary synthesis technology.

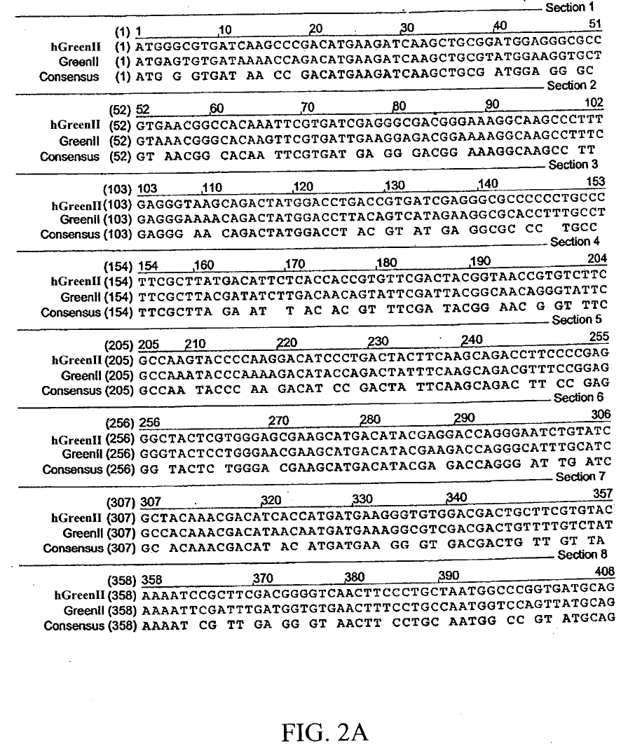

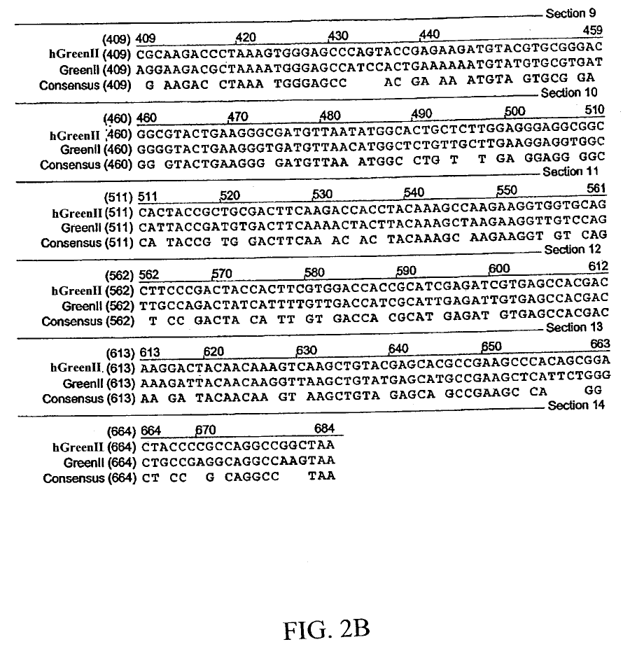

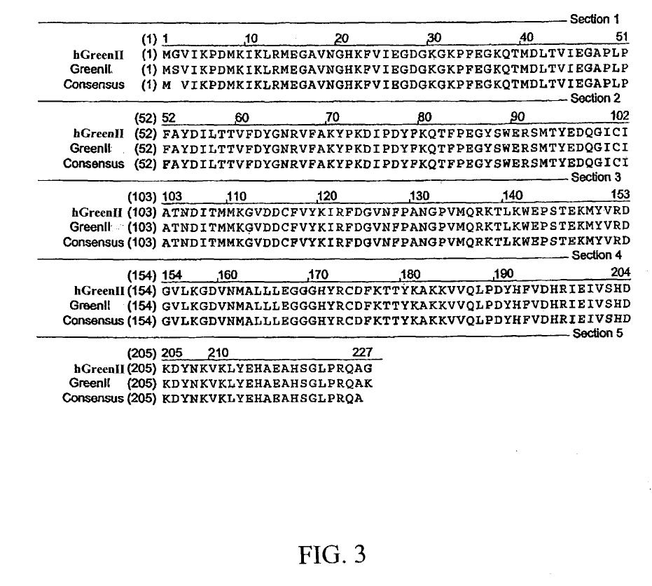

The version of the synthetic gene that was eventually synthesized is referred to herein as hGreen II. The final sequence of hGreen II has 3 vertebrate transcription factor binding sequences, whereas the parent Green II molecule contains 67 vertebrate transcription factor binding sequences. FIGS. 2A-2B show an alignment of the DNA encoding hGreen II and the parent Green II, FIG. 3 shows an alignment of the amino acids encoded by the DNA of hGreenII and the parent GreenII, and FIGS. 4A-4D show an alignment of the various DNA sequences encoding the intermediate versions of the Green II and 2M1-h8, including their respective flanking sequences.

As is illustrated in FIG. 3, there are only two amino acid differences between hGreen II and the parent Green II, at amino acid positions 2 and 227. At amino acid 2, hGreen II has a Gly (GGC), and the parent Green II has a Ser (AGT) at this same position. At this codon, the DNA sequence was changed to add a Kozak sequence for improved expression. In addition, at amino acid 227, hGreen II has a Gly (GGC), whereas Green II has a Lys (AAG). This change in the DNA sequence adds a novel NaeI restriction sequence, providing a cloning site for the creation of, e.g., fusion proteins.

### EXAMPLE 2

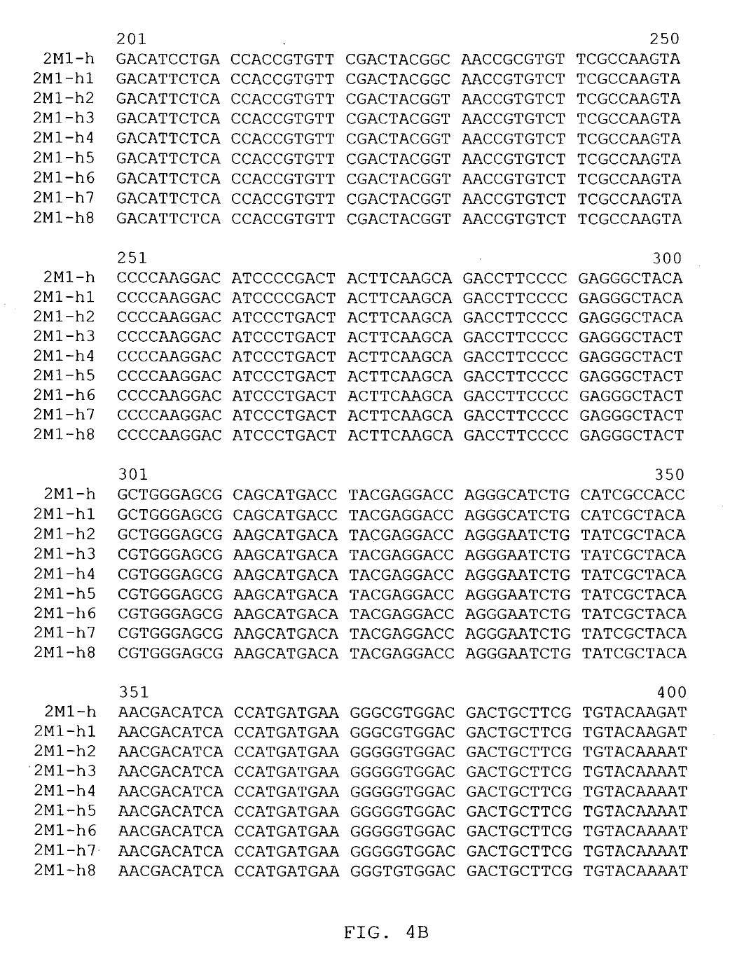

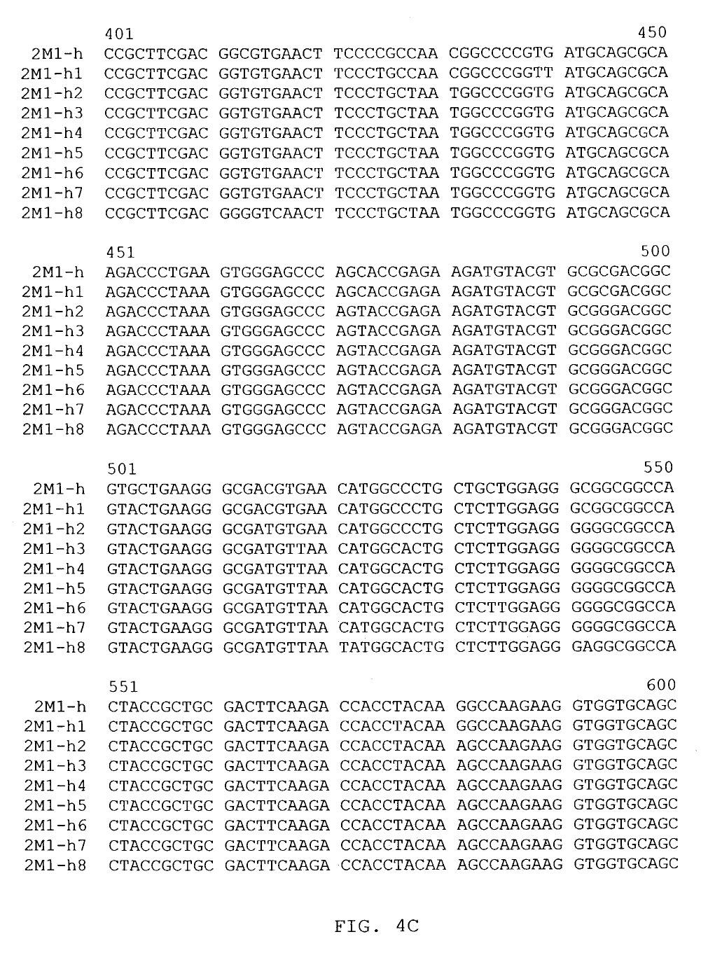

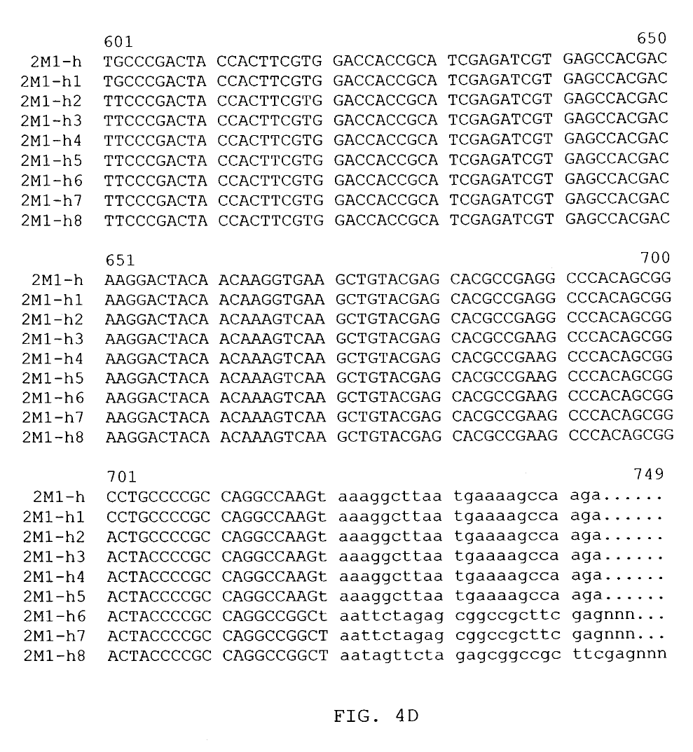

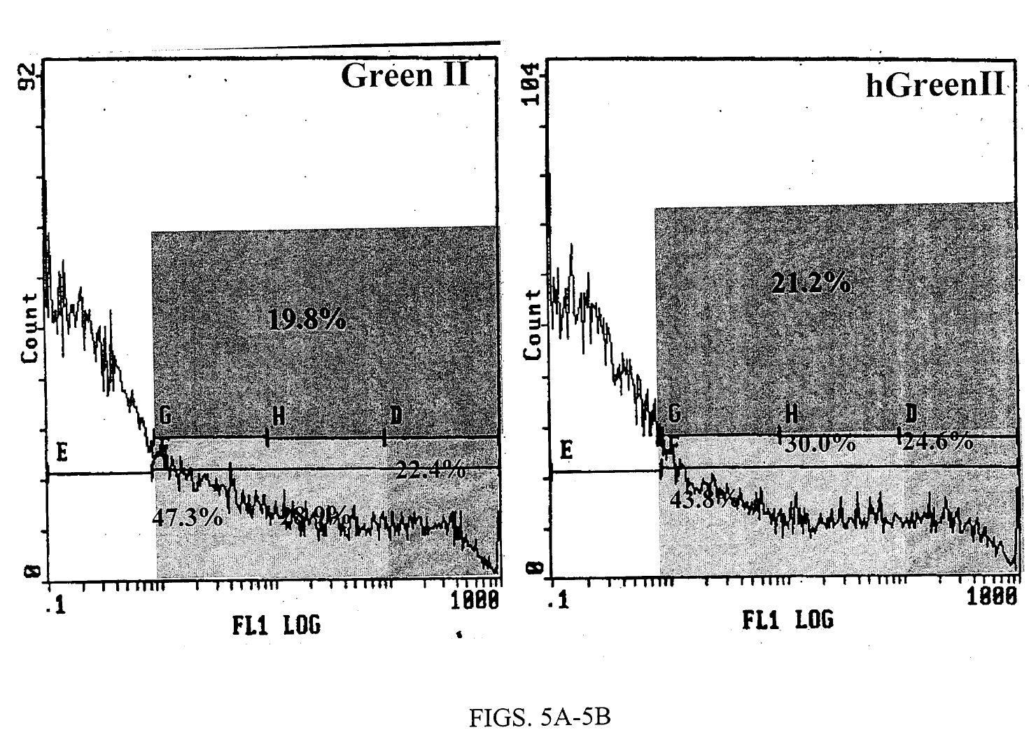

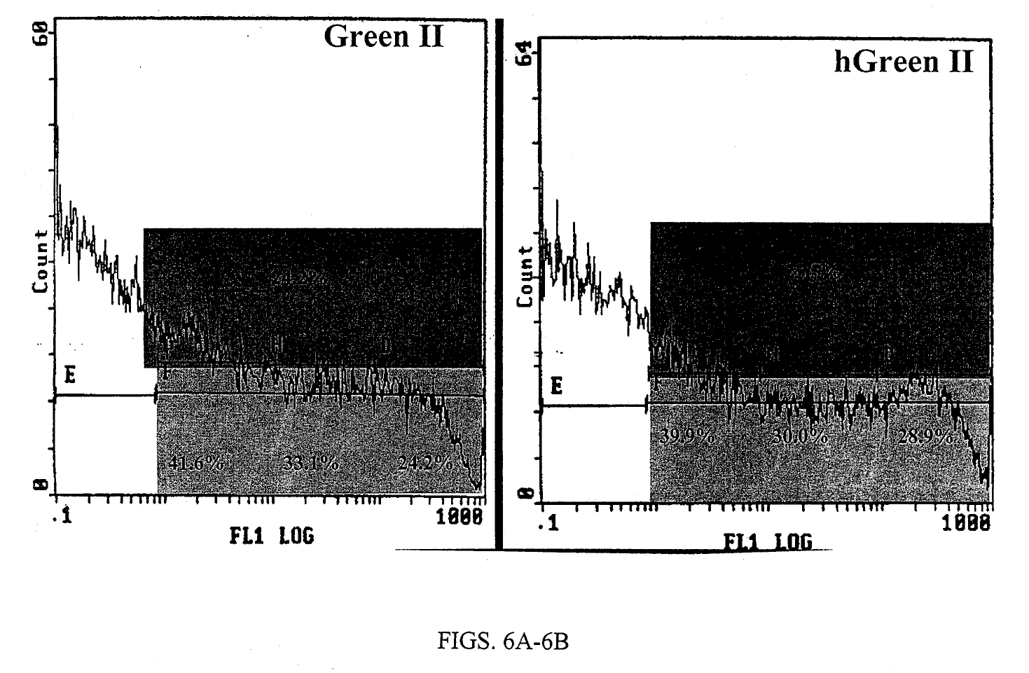

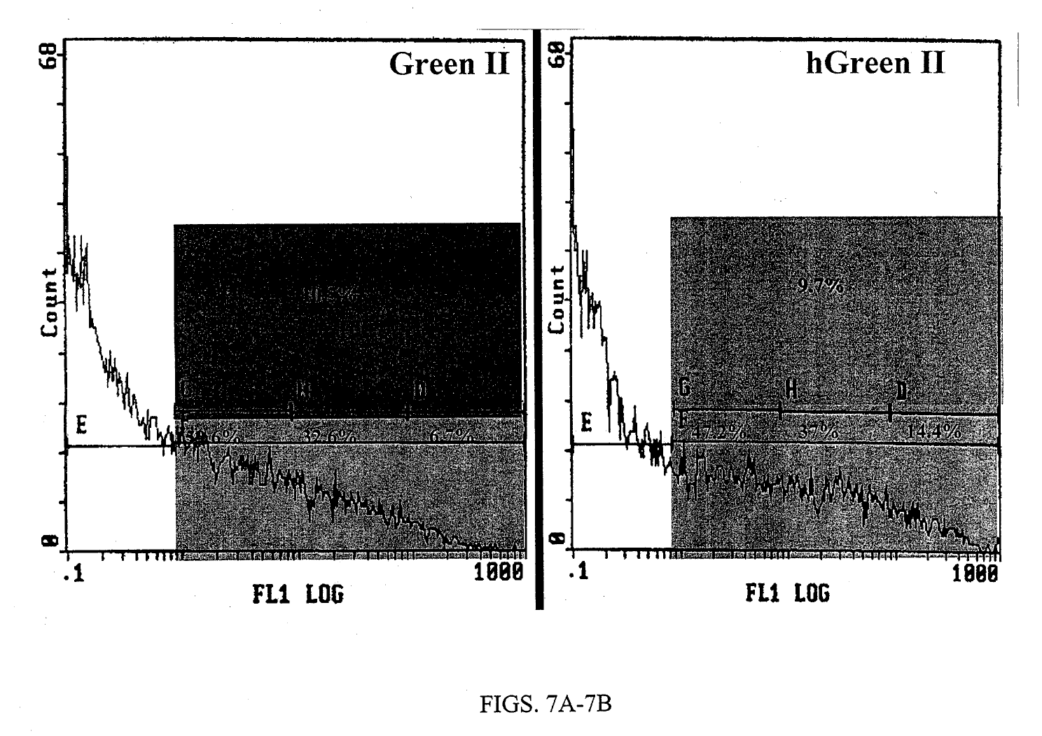

A vector construct was made by cloning the synthetic hGreen II gene into a plasmid pCl-Neo Mammalian Expression Vector (Promega Corp.). In addition, a vector construct was made by cloning the parent Green II gene into a plasmid pCI-Neo Mammalian Expression Vector (Promega Corp.) As is illustrated in FIGS. 5A-5B and 6A-6B, the hGreen II construct showed slightly higher expression in the CHO cells than did the parent Green II construct. In a first experiment using CHO cells, parent Green II showed 19.8% transfection efficiency (FIG. 5A), and hGreen II showed 21.2% transfection efficiency (FIG. 5B). In a second experiment with the CHO cells, parent Green II showed 24.2% transfection efficiency (FIG. 6A), and hGreen II showed 25.5% transfection efficiency (FIG. 6B). More importantly, the degree of fluorescence was higher in the cells transformed by the hGreen II construct. In FIG. 5A, the parent Green II, 22.4% fluoresced at 3 full logs higher than untransfected cells while FIG. 5B shows that 24.6% of the humanized Green II transformed cells fluorescenced at 3 full logs higher than untransformed cells. In FIGS. 6A and 6B, the percentage of cells that fluorescenced 3 full logs over nontransfecteded cells are 24.2% and 28.9% respectively. In NIH 3T3 cells, parent Green II showed 10.5% transfection efficiency (FIG. 7A), and hGreen II showed 9.7% transfection efficiency (FIG. 7B), in efficiency for this plasmid in this mouse cell line. However, the percentage of cells that are fluorescing at 3 logs over untransfected control is 6.7% for the parent plasmid and is 14.4% for the hGreen II, which is a 115% increase. It should be noted that such differences may be expected as neither of these are the species for which the nucleic acid sequence was optimized.

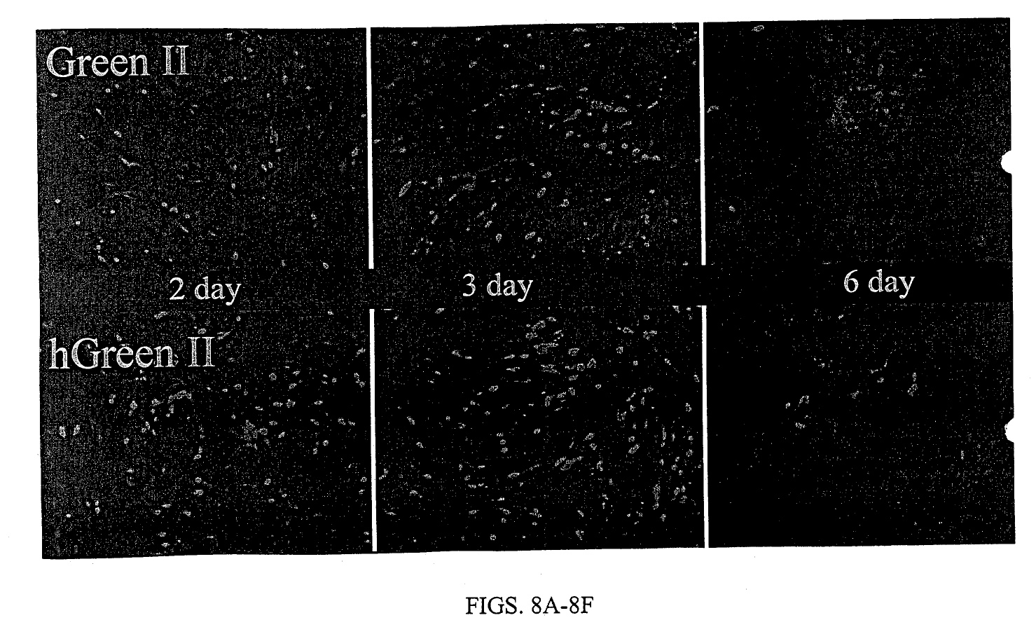

FIGS. 8A-8F show images of NIH 3T3 cells transfected with the parent Green II vector construct and the hGreen II vector construct at 2 days, 3 days, and 6 days after transfection. At each time point, NIH 3T3 cells transfected with the hGreen II vector construct show higher expression of the fluorescent protein than the NIH 3T3 cells transfected with parent Green II vector construct, consistent with FIG. 7.

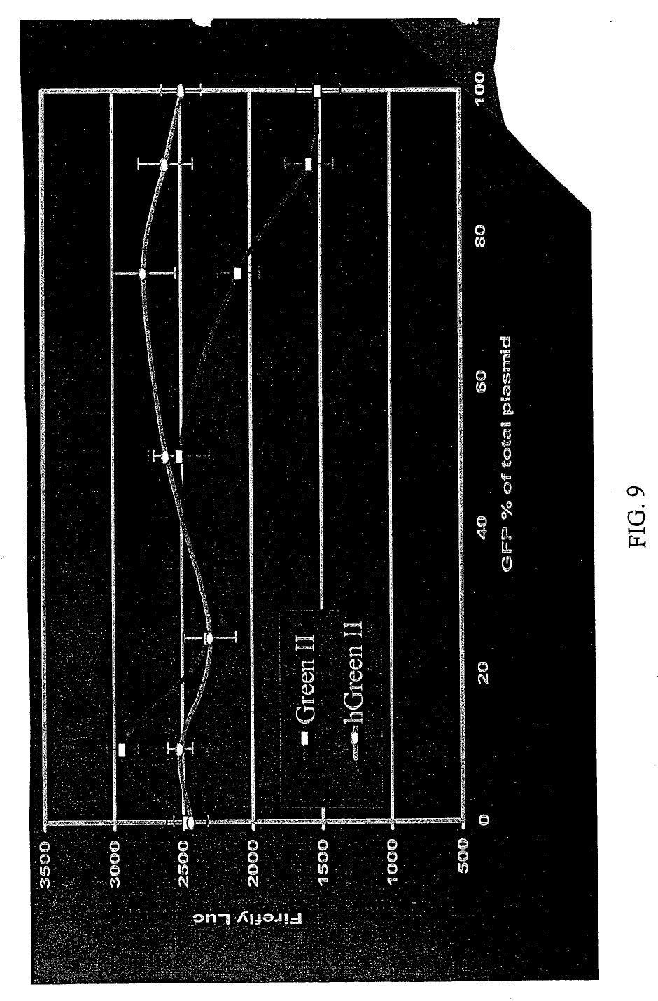

FIG. 9 is a graph showing NIH 3T3 cells transfected with an increasing concentration of the hGreen II vector construct and the parent Green II vector construct, each of which was cotransfected with a luciferase reporter. Luciferase activity is shown on the Y-axis and the relative % of GFP construct is shown on the X-axis. This experiment is an indirect measure of whether the GFP plasmid is acting as a “sink” for unproductive transcription factor binding events. If the cellular transcription factors are binding at a high rate to the GFP plasmid, than luciferase expression will be impaired. This figure shows that in the presence of hGreen II, luciferase activity is relatively stable, regardless of how much GFP is present. In the presence of increasing levels of the parent Green II, luciferase expression is impaired. This finding is important if an investigator wishes to study low-expressing transcripts; a reporter that uses transcription factors unproductively will impair the results of the assay.

### BIBLIOGRAPHY

Altschul et al. (1990) J Mol Biol. 215:403.

Altschul et al. (1997) Nucl. Acids Res. 25:3389.

Ausubel, et al., (1992) Current Protocols in Molecular Biology. John Wiley & Sons, New York.

Boshart et al. (1985) Cell 41:521.

Corpet et al. (1988) Nucl. Acids Res. 16:881.

Dijkema et al. (1985) EMBO J., 4:761.

Fradkov, A. F., et al. (2000) FEBS Letters 479:127.

Gibbs, P. D. L., et al. (1994) Mol. Mar. Biol. Biotechnol. 3:307.

Gibbs, P. D. L. et al. (2000) Marine Biotechnology 2:107.

Gorman et al. (1982) Proc. Natl. Acad. Sci. USA 79:6777.

Higgins et al. (1988) Gene 73:237

Higgins et al. (1989) CABIOS 5:151.

Huang et al. (1992) CABIOS 8:155.

Johnson et al., (1998) Mol. Reprod. Devel. 50:377.

Jones et al., (1997) Mol. Cell. Biol. 17:6970.

Karlin and Altschul (1990) Proc. Natl. Acad. Sci. USA 87:2264.

Karlin and Altschul (1993) Proc. Natl. Acad. Sci. USA 90:5873.

Kim, et al. (1990) Gene 91:217

Lamb et al., (1998) Mol. Reprod. Devel. 51: 218.

Liu, H. S., et al. (1999) Biochemical & Biophysical Research Communications 260:712.

Maniatis et al., (1987) Science 236:1237.

Matz, M. V., et al. (1999) Nature Biotech 17:969.

Michael et al., (1990) EMBO. J. 9: 481.

Mizushima and Nagata (1990) Nucl. Acids Res. 18:5322.

Myers and Miller (1988) CABIOS 4:11.

Needleman and Wunsch (1970) J. Mol. Biol. 48: 443.

Ormo, M., et al. (1996) Science 273:1392.

Pearson et al. (1994) Meth. Mol. Biol. 24: 307.

Pearson and Lipman (1988) Proc. Natl. Acad. Sci. USA 85: 2444.

Smith and Waterman (1981) Adv. Appl. Math. 2: 482.

Uetsuki et al. (1989) J. Biol. Chem. 264:5791

Voss et al. (1986) Trends Biochem. Sci., 11: 287.

Yang, F., Moss, L. G., and Phillips, G. N., Jr. (1996) Nature Biotech 14:1246.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A synthetic nucleic acid molecule comprising nucleotides of a coding region for a fluorescent polypeptide having a codon composition differing at more than 25% of the codons from a parent nucleic acid sequence encoding a fluorescent polypeptide, wherein the synthetic nucleic acid molecule has at least 3-fold fewer transcription regulatory sequences relative to the average number of such sequences in the parent nucleic acid sequence.

2. The synthetic nucleic acid molecule of claim 1, wherein the transcription regulatory sequences are selected from the group consisting of transcription factor binding sequences, intron splice sequences, poly(A) addition sequences, and promoter sequences.

3. The synthetic nucleic acid molecule of claim 1, wherein the synthetic nucleic acid molecule has at least 5-fold fewer transcription regulatory sequences relative to the average number of such sequences in the parent nucleic acid sequence.

4. The synthetic nucleic acid molecule of claim 1, wherein the polypeptide encoded by the synthetic nucleic acid molecule has at least 85% sequence identity to the polypeptide encoded by the parent nucleic acid sequence.

5. The synthetic nucleic acid molecule of claim 1, wherein the polypeptide encoded by the synthetic nucleic acid molecule has at least 90% contiguous sequence identity to the polypeptide encoded by the parent nucleic acid sequence.

6. The synthetic nucleic acid molecule of claim 1, wherein the codon composition of the synthetic nucleic acid molecule differs from the parent nucleic acid sequence at more than 35% of the codons.

7. The synthetic nucleic acid molecule of claim 1, wherein the codon composition of the synthetic nucleic acid molecule differs from the parent nucleic acid sequence at more than 45% of the codons.

8. The synthetic nucleic acid molecule of claim 1, wherein the codon composition of the synthetic nucleic acid molecule differs from the parent nucleic acid sequence at more than 55% of the codons.

9. The synthetic nucleic acid molecule of claim 1, wherein the majority of codons which differ are ones that are preferred codons of a desired host cell.

10. The synthetic nucleic acid molecule of claim 1, wherein the synthetic nucleic acid molecule encodes a green fluorescent polypeptide.

11. The synthetic nucleic acid molecule of claim 1, wherein the synthetic nucleic acid molecule encodes a green fluorescent polypeptide that was derived from a nucleic acid molecule that was originally isolated from Montastraea cavernosa.

12. The synthetic nucleic acid molecule of claim 1, wherein the synthetic nucleic acid molecule comprises SEQ ID NO: I (hGreen II).

13. The synthetic nucleic acid molecule of claim 1, wherein the parent nucleic acid sequence encodes a green fluorescent polypeptide.

14. The synthetic nucleic acid molecule of claim 13, wherein the parent nucleic acid sequence encodes a green fluorescent polypeptide isolated from Montastraea cavernosa.

15. The synthetic nucleic acid molecule of claim 14, wherein the synthetic nucleic acid molecule encodes the amino acid sequence of SEQ. ID. NO: 2.

16. The synthetic nucleic acid molecule of claim 1, wherein the majority of codons which differ in the synthetic nucleic acid molecule are those which are employed more frequently in mammals.

17. The synthetic nucleic acid molecule of claim 1, wherein the majority of codons which differ in the synthetic nucleic acid molecule are those which are preferred codons in humans.

18. The synthetic nucleic acid molecule of claim 17, wherein the majority of codons which differ are the human codons CGC, CTG, TCT, AGC, ACC, CCA, CCT, GCC, GGC, GTG, ATC, ATT, AAG, AAC, CAG, CAC, GAG, GAC, TAC, TGC and TTC.

19. The synthetic nucleic acid molecule of claim 17, wherein the majority of codons which differ are the human codons CGC, CTG, TCT, ACC, CCA, GCC, GGC, GTC, and ATC or codons CGT, TTG, AGC, ACT, CCT, GCT, GGT, GTG and ATT.

20. The synthetic nucleic acid molecule of claim 1, wherein the majority of codons which differ in the synthetic nucleic acid molecule are those which are preferred codons in plants.

21. The synthetic nucleic acid molecule of claim 20, wherein the majority of codons which differ are the plant codons CGC, CTT, TCT, TCC, ACC, CCA, CCT, GCT, GGA, GTG, ATC, ATT, AAG, AAC, CAA, CAC, GAG, GAC, TAC, TGC and TTC.

22. The synthetic nucleic acid molecule of claim 20, wherein the majority of codons which differ are the plant codons CGC, CTT, TCT, ACC, CCA, GTC, GGA, GTC, and ATC or codons CGT, TGG, AGC, ACT, CCT, GCC, GGT, GTG and ATT.

23. The synthetic nucleic acid molecule of claim 1, wherein the synthetic nucleic acid molecule is expressed in a mammalian host cell at a level which is greater than that of the parent nucleic acid sequence.

24. The synthetic nucleic acid molecule of claim 1, wherein the synthetic nucleic acid molecule has an increased number of CTG or TTG leucine-encoding codons.

25. The synthetic nucleic acid molecule of claim 1, wherein the synthetic nucleic acid molecule has an increased number of GTG or GTC valine-encoding codons.

26. The synthetic nucleic acid molecule of claim 1, wherein the synthetic nucleic acid molecule has an increased number of GGC or GGT glycine-encoding codons.

27. The synthetic nucleic acid molecule of claim 1, wherein the synthetic nucleic acid molecule an increased number of ATC or ATT isoleucine-encoding codons.

28. The synthetic nucleic acid molecule of claim 1, wherein the synthetic nucleic acid molecule has an increased number of CCA or CCT proline-encoding codons.

29. The synthetic nucleic acid molecule of claim 1, wherein the synthetic nucleic acid molecule has an increased number of CGC or CGT arginine-encoding codons.

30. The synthetic nucleic acid molecule of claim 1, wherein the synthetic nucleic acid molecule has an increased number of AGC or TCT serine-encoding codons.

31. The synthetic nucleic acid molecule of claim 1, wherein the synthetic nucleic acid molecule has an increased number of ACC or ACT threonine-encoding codons.

32. The synthetic nucleic acid molecule of claim 1, wherein the synthetic nucleic acid molecule has an increased number of GCC or GCT alanine-encoding codons.

33. The synthetic nucleic acid molecule of claim 1, wherein the codons in the synthetic nucleic acid molecule which differ encode the same amino acids as the corresponding codons in the parent nucleic acid sequence.

34. The synthetic nucleic acid molecule of claim 1, wherein the synthetic nucleic acid molecule is expressed at a level which is at least 110% of that of the parent nucleic acid sequence in a cell or cell extract under identical conditions.

35. The synthetic nucleic acid molecule of claim 1, wherein the polypeptide encoded by the synthetic nucleic acid molecule is identical in amino acid sequence to the polypeptide encoded by the parent nucleic acid sequence.

36. The nucleic acid molecule of claim 1, wherein the synthetic nucleic acid molecule comprises SEQ ID NO:1 (hGreen II), nucleotides 22 to 702 of SEQ ID NO:3 (2M1-h), nucleotides 22 to 702 of SEQ ID NO:5 (2M1-hl), nucleotides 22 to 702 of SEQ ID NO:7 (2M1-h2), nucleotides 22 to 702 of SEQ ID NO:9 (2M1-h3), nucleotides 22 to 702 of 5 SEQ ID NO:11 (2M1-h4), nucleotides 22 to 702 of SEQ ID NO:13 (2M1-h5), nucleotides 39 to 719 of SEQ ID NO:15 (2M1-h6), or nucleotides 38 to 718 of SEQ ID NO: 17 (2M1-h7).

37. A vector construct comprising a synthetic vector backbone having at least 3-fold fewer transcriptional regulatory sequences relative to a parent vector backbone; and the nucleic acid molecule of claim 1.

38. A plasmid comprising the synthetic nucleic acid molecule of claim 1.

39. An expression vector comprising the synthetic nucleic acid molecule of claim 1 linked to a promoter functional in a cell.

40. The expression vector of claim 39, wherein the synthetic nucleic acid molecule is operatively linked to a Kozak consensus sequence.

41. The expression vector of claim 39, wherein the promoter is functional in a mammalian cell.

42. The expression vector of claim 39, wherein the promoter is functional in a human cell.

43. The expression vector of claim 39, wherein the promoter is functional in a plant cell.

44. The expression vector of claim 39, wherein the expression vector further comprises a multiple cloning site.

45. The expression vector of claim 44, wherein the multiple cloning site is positioned between the promoter and the synthetic nucleic acid molecule.

46. The expression vector of claim 44, wherein the multiple cloning site is positioned downstream from the synthetic nucleic acid molecule.

47. A host cell comprising the expression vector of claim 39.

48. A kit comprising, in a suitable container, the expression vector of claim 39.

49. A polynucleotide which hybridizes under at least low stringency hybridization conditions to the synthetic nucleic acid molecule comprising SEQ ID NO: 1 (hGreen II), nucleotides 22 to 702 of SEQ ID NO:3 (2M1-h), nucleotides 22 to 702 of SEQ ID NO:5 (2M1-h1), nucleotides 22 to 702 of SEQ ID NO:7 (2M1-h2), nucleotides 22 to 702 of SEQ ID NO:9 (2M1-h3), nucleotides 22 to 702 of SEQ ID NO:11 (2M1-h4), nucleotides 22 to 702 of SEQ ID NO:13 (2M1-h5), nucleotides 39 to 719 of SEQ ID NO:15 (2M1-h6), or nucleotides 38 to 718 of SEQ ID NO: 17 (2M 1-h7), or the complement thereof.

50. The polynucleotide of claim 49, wherein the polynucleotide hybridizes under at least low stringency hybridization conditions to the synthetic nucleic acid molecule comprising SEQ. ID. NO: 1 (hGreen II), or the complement thereof.

51. A method to prepare a synthetic nucleic acid molecule comprising an open reading frame, comprising:
a) altering a plurality of transcription regulatory sequences in a parent nucleic acid sequence which encodes a fluorescent polypeptide to yield a synthetic nucleic acid molecule which has at least 3-fold fewer transcription regulatory sequences relative to the parent nucleic acid sequence; and
b) altering greater than 25% of the codons in the synthetic nucleic acid sequence which has a decreased number of transcription regulatory sequences to yield a further synthetic nucleic acid molecule, wherein the codons which are altered do not result in an increased number of transcription regulatory sequences, wherein the further synthetic nucleic acid molecule encodes a polypeptide with at least 85% amino acid sequence identity to the polypeptide encoded by the parent nucleic acid sequence.

52. A method to prepare a synthetic nucleic acid molecule comprising an open reading frame, comprising:
a) altering greater than 25% of the codons in a parent nucleic acid sequence which encodes a fluorescent polypeptide to yield a codon-altered synthetic nucleic acid molecule, and
b) altering a plurality of transcription regulatory sequences in the codon-altered synthetic nucleic acid molecule to yield a further synthetic nucleic acid molecule which has at least 3-fold fewer transcription regulatory sequences relative to a synthetic nucleic acid molecule with codons which differ from the corresponding codons in the parent nucleic acid sequence, and wherein the further synthetic nucleic acid molecule encodes a polypeptide with at least 85% amino acid sequence identity to the fluorescent polypeptide encoded by the parent nucleic acid sequence.

53. The method of claim 51 or 52, wherein the transcription regulatory sequences are selected from the group consisting of transcription factor binding sequences, intron splice sequences, poly(A) addition sequences, enhancer sequences and promoter sequences.

54. The method of claim 51 or 52 wherein the parent nucleic acid sequence encodes a green fluorescent polypeptide.

55. The method of claim 51 or 52, wherein the parent nucleic acid sequence encodes a green fluorescent polypeptide isolated from Montastraea cavernosa.

56. The method of claim 51 or 52, wherein the synthetic nucleic acid molecule hybridizes under medium stringency hybridization conditions to the parent nucleic acid sequence.

57. The method of claim 51 or 52, wherein the codons which are altered encode the same amino acid as the corresponding codons in the parent nucleic acid sequence.

58. A synthetic nucleic acid molecule which is the further synthetic nucleic acid molecule prepared by the method of claim 52 or 53.

59. The method of claim 51 or 52, further comprising altering the further synthetic nucleic acid molecule to encode a polypeptide having at least one amino acid substitution relative to the polypeptide encoded by the parent nucleic acid sequence.

60. The method of claim 51 or 52, wherein the altering of transcription regulatory sequences introduces less than 1% amino acid substitutions to the polypeptide encoded by the synthetic nucleic acid molecule.

61. A method for preparing at least two synthetic nucleic acid molecules which are codon distinct versions of a parent nucleic acid sequence which encodes a fluorescent polypeptide, comprising:
a) altering a parent nucleic acid sequence to yield a synthetic nucleic acid molecule having an increased number of a first plurality of codons that are employed more frequently in a selected host cell relative to the number of those codons in the parent nucleic acid sequence; and
b) altering the parent nucleic acid sequence to yield a further synthetic nucleic acid molecule having an increased number of a second plurality of codons that are employed more frequently in the host cell relative to the number of those codons in the parent nucleic acid sequence, wherein the first plurality of codons is different than the second plurality of codons, and wherein the synthetic and the further synthetic nucleic acid molecules encode the same polypeptide.

62. The method of claim 61, further comprising altering a plurality of transcription regulatory sequences in the synthetic nucleic acid molecule, the further synthetic nucleic acid molecule, or both, to yield at least one yet further synthetic nucleic acid molecule which has at least 3-fold fewer transcription regulatory sequences relative to the synthetic nucleic acid molecule, the further synthetic nucleic acid molecule, or both.

63. The method of claim 61, further comprising altering at least one codon in the first synthetic sequence to yield a first modified synthetic sequence which encodes a polypeptide with at least one amino acid substitution relative to the polypeptide encoded by the first synthetic nucleic acid sequence.

64. The method of claim 61, further comprising altering at least one codon in the second synthetic sequence to yield a second modified synthetic sequence which encodes a polypeptide with at least one amino acid substitution relative to the polypeptide encoded by the first synthetic nucleic acid sequence.
